(19) United States
(12) Patent Application Publication
(10) Pub. No.: US 2020/0033363 A1
(43) Pub. Date: Jan. 30, 2020

(54) SCREENING METHODS

(71) Applicant: GARVAN INSTITUTE OF MEDICAL RESEARCH, Darlinghurst, New South Wales (AU)

(72) Inventors: Daniel CHRIST, Camperdown, New South Wales (AU); Mary CHRISTIE, Sydney, New South Wales (AU)

(21) Appl. No.: 16/497,788

(22) PCT Filed: Mar. 27, 2018
(86) PCT No.: PCT/AU2018/050283

§ 371 Date: Sep. 25, 2019

(30) Foreign Application Priority Data
Mar. 27, 2017 (AU) 2017901098

(51) Int. Cl. G01N 33/68; C07K 16/22; C07K 16/40; G01N 33/531; C07K 1/22

(52) U.S. Cl. CPC G01N 33/6854; C07K 16/22; C07K 16/40; G01N 33/531; C07K 1/22; G01N 2500/04; C07K 2317/21; C07K 2317/33; C07K 2317/35; C07K 2317/565; C07K 2317/92; C07K 2317/10

(57) ABSTRACT

The present disclosure generally relates to proteins containing immunoglobulin light chain variable region homodimers, to methods of providing such proteins and to various uses of such proteins. In one example, the disclosure provides a method for screening a plurality of immunoglobulin homodimers, each homodimer comprising two identical light chain variable domain monomers, wherein screening of the immunoglobulin homodimers comprises identifying homodimers that are capable of binding specifically to an antigen to form an immunoglobulin-antigen complex.

### RELATED APPLICATIONS

This application is a 35 U.S.C. § 371 filing of International Patent Application No. PCT/AU2018/050283, filed Mar. 27, 2018, which claims the benefit of Australian Patent Application No. 2017901098, filed Mar. 27, 2017, the entire disclosures of which are hereby incorporated herein by reference.

### TECHNICAL FIELD

The present disclosure generally relates to proteins containing immunoglobulin light chain variable region homodimers, to methods of providing such proteins and to various uses of such proteins.

### BACKGROUND

First discovered in 1975 by Kohler and Milstein (Kohler and Milstein, 1975), monoclonal antibodies (mAbs) were quickly recognised as a biological tool which could have wide ranging applicability as research agents, diagnostic/prognostic reagents, industrial reagents as well as therapeutic agents. This broad ranging applicability arises from the ability of antibodies (and more generally, proteins containing immunoglobulin domains) to bind with high specificity and affinity to a target molecule. As is known in the art, many types of antibodies of defined specificity, affinity and antibody class have been manufactured, which bind to a variety of specific target molecules.

One class of manufactured antibodies are single domain antibodies (or “nanobodies”). Unlike standard human antibodies which are exclusively expressed as paired species containing both heavy and light chains, single domain antibodies are a unique type of functional antibodies that contain either only the heavy chain variable region or only the light chain variable region, while preserving the antigen-binding properties of conventional antibodies.

Single domain antibodies are able to demonstrate high specificity and affinity similar to standard IgG antibodies but are much smaller in size. Properties such as this have rendered single domain antibodies useful in a number of applications, e.g. for biosensor, diagnostic and therapeutic applications. Although single domain antibodies exist as monomers, they can dimerize to form dimers. For example, light chain dimers known as Bence-Jones proteins (Bernier and Putnam, 1963) have previously been described. Studies have shown that light chain antibody dimers are able to bind to a protein antigen but with poor affinity (Edmundson et al. 1993; Edmundson and Manion, 1998).

### SUMMARY

The present inventors have shown for the first time that a method as disclosed herein can be used to provide one or more immunoglobulin homodimers that are able to bind to an antigen target with high specificity and affinity. In particular, the inventors have demonstrated that an immunoglobulin homodimer comprising identical light chain variable region (V_{L}) monomers is able to bind to an antigen target with superior specificity and affinity compared to previously described immunoglobulin homodimers comprising either V_{H} monomers or V_{L} monomers. These findings provide the basis for an effective and improved method of producing, screening and selecting for immunoglobulin homodimers that are highly specific towards any target of interest. The inventors have also demonstrated that the immunoglobulin homodimers described herein bind with high specificity and affinity to an antigen that is either symmetrical or asymmetrical with a 2:1 stoichiometric ratio.

Accordingly, the present disclosure provides a method for screening a plurality of immunoglobulin homodimers, each homodimer comprising two identical light chain variable domain monomers, wherein screening of the immunoglobulin homodimers comprises identifying homodimers that are capable of binding specifically to an antigen to form an immunoglobulin-antigen complex.

The present disclosure also provides a plurality of immunoglobulin homodimers which are prepared by providing a plurality of light chain variable domain monomers under conditions that allow any of the light chain variable domain monomers that are capable of dimerizing, to dimerize. The plurality of light chain variable domain monomers may comprise a library of light chain variable domains, wherein the amino acid sequence of two or more light chain variable domains in the library varies.

The methods disclosed herein may comprise expressing each light chain variable domain monomer in a bacteriophage that is capable of presenting multiple copies of the light chain variable domain monomer on its surface.

The methods disclosed herein may further comprise performing an affinity screening step and a dimerization screening step. The affinity screening step may be performed before the dimerization screening step, or vice versa. In one example, the affinity screening step is performed before the dimerization screening step.

The affinity screening step may comprise contacting the plurality of immunoglobulin homodimers with the antigen and selecting those immunoglobulin homodimers that bind specifically to the antigen.

The dimerization screening step may comprise determining the stoichiometric ratio of immunoglobulin monomer to antigen in the immunoglobulin-antigen complex.

The methods disclosed herein may comprise selecting an immunoglobulin homodimer when the stoichiometric ratio of immunoglobulin monomer to antigen in the immunoglobulin-antigen complex is about 2:1. In one example, the stoichiometric ratio of immunoglobulin monomer to antigen in the immunoglobulin-antigen complex of about 2:1 may be determined by characterising the structure of the immunoglobulin-antigen complex. For example, the stoichiometric ratio may be determined by determining the crystal structure of the immunoglobulin-antigen complex.

Alternatively or in addition, the stoichiometric ratio of immunoglobulin monomer to antigen in the immunoglobulin-antigen complex may be determined by (i) measuring the quantity of any two or more of the immunoglobulin homodimer, antigen and immunoglobulin-antigen complex in a mixture containing the immunoglobulin homodimer and the antigen and (ii) comparing the relative quantities of any two or more of the immunoglobulin homodimer, antigen and immunoglobulin-antigen complex measured in (i) to determine the stoichiometric ratio of immunoglobulin monomer to antigen in the immunoglobulin-antigen complex.

The determination of the stoichiometric ratio of immunoglobulin monomer to antigen in the immunoglobulin-antigen may further comprise measuring the quantity of unbound immunoglobulin monomer, unbound antigen, and/or unbound immunoglobulin homodimer in the mixture.

The methods disclosed herein may further comprise separating the immunoglobulin homodimer, antigen and immunoglobulin-antigen complex. For example, the immunoglobulin homodimer, antigen and immunoglobulin-antigen complex may be separated by chromatography. In one example, the chromatography is size exclusion chromatography, though other chromatography methods may equally be used.

The methods of determining the stoichiometric ratio of immunoglobulin to antigen may be determined using an apparatus comprising particles or a matrix for separating compounds of different molecular weight, whereby said compounds are eluted from said apparatus in order of descending molecular weight. In one example, the methods may comprise contacting the apparatus with a mixture containing a predetermined amount of the light chain variable domain monomer or the immunoglobulin homodimer, and containing a predetermined amount of the antigen; and eluting from the apparatus the light chain variable domain monomer and/or the immunoglobulin homodimer; eluting from the apparatus any unbound antigen; and/or eluting from the apparatus the immunoglobulin-antigen complex.

The methods may comprise incubating the mixture for at least 1 hour at room temperature prior to contact with the apparatus.

The present disclosure also provides a method wherein the determination of the stoichiometric ratio of immunoglobulin monomer to antigen in the immunoglobulin-antigen complex comprises measuring by light scattering. Thus, the methods may comprise the use of a light scattering photometer. The light scattering photometer may additionally be used with an interferometric refractometer.

In any specific example of the methods disclosed herein, the affinity screening step may be performed using an optical analytical technique for measuring biomolecular interactions. For example, the methods may comprise any one of dual polarisation interferometry, static light scattering, dynamic light scattering, surface plasmon resonance, fluorescence polarisation/anisotropy, fluorescence correlation spectroscopy or nuclear magnetic resonance.

The present disclosure also provides a method wherein the affinity screening step comprises measuring the binding kinetics, binding specificity, rates of association and dissociation, or concentration of bound components, of the immunoglobulin-antigen complex.

The affinity screening step may comprise measuring the association constant (Kₐ), dissociation constant (K_{d}) and/or equilibrium binding constant (K_{D}).

The methods disclosed herein may further comprise selecting a homodimer having nanomolar affinity for the antigen. The methods may further comprise isolating a homodimer having nanomolar affinity for the antigen; providing a polynucleotide encoding the isolated homodimer; expressing the isolated homodimer; and/or formulating the isolated homodimer for administration as a pharmaceutical composition.

The methods disclosed herein may further comprise selecting a homodimer which binds to a planar surface on the antigen or a cleft within the surface of the antigen.

The methods disclosed herein may further comprise selecting a homodimer which covers a surface area of the antigen of over 600 Å². For example, the methods may comprise selecting a homodimer which covers a surface area of the antigen of over 700 Å², or over 800 Å², or over 850 Å², or over 900 Å², or over 950 Å², or over 1000 Å².

The light chain variable domain monomers of the homodimer disclosed herein may comprise one or more tyrosine residues in one or more complementarity determining regions (CDRs) thereof. The one or more tyrosine residues may provide conformational flexibility in the one or more CDRs.

The methods disclosed herein may comprise providing the light chain variable domain monomers by expressing a polynucleotide comprising human light chain variable region V and J segments. Polynucleotide heterogeneity may be introduced into the variable region V and J segments in order to provide different light chain variable region sequences.

The present disclosure also provides a method wherein variation in the plurality of immunoglobulin homodimers is provided by performing mutagenesis. For example, the variation may be provided by performing site directed mutagenesis.

The methods disclosed herein may further comprise selecting a homodimer having different amino acid residues which contact the antigen in each monomer of the homodimer. As disclosed herein, identical light chain variable region sequences in an immunoglobulin homodimer may contact an antigen at different amino acids in each variable region monomer.

The present disclosure also provides a method wherein the antigen is a symmetrical antigen or an asymmetrical antigen. The immunoglobulin homodimers disclosed herein have been shown to be particularly advantageous because of their ability to bind to different epitopes on a given antigen, such as an asymmetrical antigen. In addition, the immunoglobulin homodimers disclosed herein have been shown to be particularly advantageous because of their ability to bind to particular epitopes on a symmetrical antigen. For example, an immunoglobulin homodimer as disclosed herein may bind within a cleft formed within two symmetrically opposed regions of a symmetrical antigen. Without being bound by theory, this may contribute additionally to the high binding affinities demonstrated herein of immunoglobulin homodimers to symmetrical antigens.

The present disclosure also provides a method wherein the symmetrical antigen or asymmetrical antigen is a physiological signalling molecule. In one example, the signalling molecule may be a VEGF family member protein. For example, the signalling molecule may be any of VEGFA, VEGFB, VEGFC, VEGFD, VEGFE and PlGF, including any isoform of any of these.

The present disclosure also provides a method wherein the immunoglobulin homodimer is fused to an Fc region. One or more immunoglobulin homodimers may be fused to an Fc region. Thus, for example, two immunoglobulin homodimers may be fused to an Fc region to form an IgG-like molecule. When two or more immunoglobulin homodimers are fused to an Fc region, each immunoglobulin homodimer may be the same or different. Thus, for example, two identical immunoglobulin homodimers may be fused to an Fc region to form an IgG-like molecule. Alternatively, two different immunoglobulin homodimers may be fused to an Fc region to form an IgG-like molecule that is capable of binding to two different antigens. Thus, the methods disclosed herein may comprise fusing two different immunoglobulin homodimers to an Fc region to form a bispecific IgG-like molecule.

The present disclosure also provides a method wherein the immunoglobulin homodimer is fused to a stabilizing agent. The stabilizing agent may be any agent that increases the stability, efficacy and/or serum half-life of the immunoglobulin homodimer. Many such stabilizing agents are known in the art (see, for example, U.S. Pat. No. 6,277,375). In one example, the stabilizing agent is or comprises an Fc domain. In another example, the stabilizing agent is or comprises polyethylene glycol (PEG).

The methods disclosed herein may further comprise isolating and/or purifying an immunoglobulin homodimer as disclosed herein.

The present disclosure also provides a pharmaceutical composition comprising the isolated and/or purified immunoglobulin homodimer, and a pharmaceutically acceptable carrier or diluent.

The present disclosure also provides an immunoglobulin homodimer identified and/or selected by any of the methods disclosed herein.

The present disclosure also provides a kit comprising an immunoglobulin homodimer identified and/or selected by any of the methods disclosed herein. The kit may further comprise instructions for use of the immunoglobulin homodimer.

The present disclosure is not to be limited in scope by the specific embodiments described herein, which are intended for the purpose of exemplification only. Functionally equivalent products, compositions and methods are clearly within the scope of the invention, as described herein.

### BRIEF DESCRIPTION OF DRAWINGS

The following figures form part of the present specification and are included to further demonstrate certain aspects of the present disclosure. The disclosure may be better understood by reference to one or more of these figures in combination with the detailed description of specific embodiments presented herein.

FIGS. 1A-1C show the evolution of rearranging immunoglobulin antigen receptors. (FIG. 1A) Chain structure of modern day heterodimeric antigen receptors (IgG antibody isotype shown). The molecule is comprised of two heavy chains (each containing four Ig domains, in dark grey (blue)) and two light chains (each containing two Ig domains, in light grey (cyan)) (Bork et al. 1994). Hypervariable complementarity determining regions (CDR), which mediate contact with antigen, are highlighted. (FIG. 1B) Gene duplication and diversification events are believed to have converted a homodimeric immunoglobulin (Ig) format (lower component, darker grey (tan)) to the heterodimeric format (light grey/dark grey (cyan/blue)) observed in the B-lymphocyte (B-cell receptor, antibodies) and T-lymphocyte (T-cell receptor) based adaptive immune systems of jawed vertebrates. (FIG. 1C) Condensed maximum likelihood phylogeny of immunoglobulin heavy and light chains of vertebrates. Branches are labelled with bootstrap values from 100 replicates. The scale bar indicates the mean number of substitutions per site.

FIG. 2 shows the method used for phage library construction. Genes encoding O12/O2/DPK9 (V-) and Jκ1 (J-) segments were diversified at hypervariable CDR positions (CDR1: 28, 30-32; CDR2: 50-51, 53; CDR3: 91-94, 96; numbering according to Kabat). Combinatorial diversity was generated through recombination of the segments by splice overlap extension PCR.

FIGS. 3A-3C show the molecular weight of the immunoglobulin homodimers, HEL antigen and immunoglobulin-antigen complex measured using size-exclusion chromatography coupled to multi-angle laser light scattering of the Ig-HEL complexes. (FIG. 3A) Elution profiles for Ig5 (black; left peak), HEL (grey; right peak) and their complex (red; middle peak) reveal molecular weight estimates of the complex to be consistent with a 2:1 stoichiometry of Ig:HEL. (FIG. 3B) Elution profiles for Ig12 (black; left peak), HEL (grey; right peak) and their complex (red; middle peak) similarly reveal a 2:1 stoichiometry for the Ig:HEL complex. (FIG. 3C) Amino acid sequences of Ig5 and Ig12 with CDR regions underlined: CDR1 (purple), CDR2 (green) and CDR3 (blue); amino acids within the CDRs that are diversified in the library are highlighted in red (for Ig5: D, second A, third A and fourth A in CDR1; A, first S and Y in CDR2; D, G, Y, Y and A in CDR3; for Ig12: D, second A, S and second D in CDR1; first S, D and Y in CDR2; Y, second Y, M, I and S in CDR3).

FIGS. 4A-4B show the method used to analyse binding kinetics of the immunoglobulin-antigen complexes. Hen egg white lysozyme antigen was immobilized on streptavidin biosensors using biotinylated HyHEL-10 (Ig5) and HyHEL-5 (Ig12) capture antibodies in an Fab format using a Forte Bio bio-layer interferometry system. (FIG. 4A) Ig5 (at 2000 nM (top line), 1000 nM (middle line) and 500 nM concentrations (bottom line)) (kₐ=4.3·104 M⁻¹s⁻¹; k_{d}=5.7·10⁻³ s⁻¹; K_{D}=1.3·10⁻⁷ M). (FIG. 4B) Ig12 (at 1000 nM (top line), 750 nM (middle line) and 375 nM concentrations (bottom line)) (kₐ=8.4·10⁴ M⁻¹s⁻¹; k_{d}=2.5·10⁻³ s⁻¹; K_{D}=3.0·10⁻⁸ M).

FIG. 5 shows the Biolayer interferometry of isolated V_{L}D9 homodimer to immobilized biotinylated VEGF (left) at varying concentrations and the ability of Avastin mAb to bind to antigenVEGF in complex with V_{L}D9 homodimer (right).

FIGS. 6A-6D show the structure of the Ig5 homodimeric antigen receptor in complex with antigen: evidence for conformational diversity. (FIG. 6A) Homodimer of Ig domains (tan cartoon with CDR1, CDR2 and CDR3 loops colored in purple, green and blue respectively) bound to a single HEL molecule (grey cartoon and surface). The symmetry axis is indicated (dashed line). (FIG. 6B) Antigen binding site (superposed Ig protomers; chains C/D): multiple side chain conformations are observed at positions Y53 and Y94. Electron density (mesh) is contoured at 1 standard deviation above average (sigma-A weighted 2mFo-DFc map). (FIG. 6C) Antigen binding site 1 (left hand side). (FIG. 6D) Antigen binding site 2 (right hand side). Hydrogen bonds are highlighted; distinct contact networks are observed for each Ig subunit.

FIGS. 7A-7D show the structure of the Ig12 homodimeric antigen receptor in complex with antigen: evidence for selective recruitment. (FIG. 7A) Homodimer of Ig domains (tan cartoon with CDR1, CDR2 and CDR3 colored in purple, green and blue respectively) bound to a single HEL molecule (grey cartoon and surface). The symmetry axis is indicated (dashed line). (FIG. 7B) HEL contact footprint mapped onto the surface of the Ig12 dimer (viewed down its two-fold axis as indicated). The contact surfaces of the two Ig domains are coloured light blue and purple. The CDR2 regions are indicated and highlight the selective use of otherwise identical surface loops. (FIG. 7C) Antigen binding site 1 (left hand side). (FIG. 7D) Antigen binding site 2 (right hand side). Hydrogen bonds are highlighted; distinct contact networks are observed for each Ig subunit.

FIG. 8 shows the contact map of the surface residues of the two protomers of Ig5 (top panel) or Ig12 (bottom panel) with the contact the surface of antigen HEL. Protomers are distinguished by color (shades of purple or orange respectively). Light shades indicate hydrophobic interactions. Darker shades indicate hydrogen bonding interactions or salt bridges. Contact information was derived from analysis output by PDBePISA

FIGS. 9A-9B show additional perspectives of Ig5_{2—}HEL complex and Ig12₂-HEL complex interactions. (FIG. 9A) Superposition of Ig12 protomers. In contrast to what is observed for Ig5 (FIG. 6B), side chain conformations are broadly conserved within the Ig12 antigen binding site (despite different contacts with the asymmetric HEL surface). (FIG. 9B) HEL contact footprint mapped onto the surface of the Ig5 dimer, as viewed down its two-fold axis, reveals different contact networks of the Ig subunits, similarly to what is observed for the Ig12 structure (FIG. 7B).

FIG. 10 shows the accessible surface areas buried by contact with ligand. Percentages of accessible surface areas buried by ligand contact are displayed for residues involved in ligand binding. These are plotted for Ig12 and Ig5 (top), and for two MCG structures containing the same ligand either soaked (PDB code 1MCF) or co-crystallized (PDB code 1MCH). The MCG structures employ some contacts (black bars), in positions outside those normally observed in V_{H}-V_{L} ligand complexes.

FIGS. 11A-11C show molecular mimicry at a conserved interface. Comparison of D11.15-PEL and Ig122-HEL structures. (FIG. 11A) The D11.15-PEL structure (PDB ID 1JHL) comprises the classic heterodimer pairing of heavy chain (blue) and light chain (cyan) bound to lysozyme (grey cartoon and surface). The active site cleft is indicated (*). (FIG. 11B) Ig122-HEL structure, viewed from the same perspective as displayed in panel a. (FIG. 11C) Superposition of lysozyme molecules reveals the CDR3 loops of the D11.15 and Ig12 components (colored as in panels a and b) to be contacting equivalent surface areas of the lysozyme antigens.

FIGS. 12A-12D show epitope preferences of Ig receptor formats. (FIG. 12A) Ig5 and Ig12 homodimers (tan cartoons) target flat epitopes on the HEL surface (grey cartoon and surface), distant from the HEL active site cleft (indicated by *). (FIG. 12B) Flat epitope surfaces apart from the active site are also observed for the model anti-HEL monoclonals HyHEL10 (PDB ID 1YQV) and HyHEL9 (PDB ID 3D9A), paired receptors formed by V_{H}-V_{L} domain assembly (blue and cyan cartoons respectively). (FIG. 12C) Structures of non-paired immunoglobulin antigen receptors of camels (VHH; PDB ID 1ZVY) and (FIG. 12D) sharks (VNAR; PDB ID 1T6V) reveal a 1:1 stoichiometry with antigen, and binding in the HEL active site cleft.

FIG. 13 shows the structure of VEGFA dimer in complex with V_{L}D9 dimer. Each V_{L} domain of V_{L}D9 homodimer contacts analogous interfaces of VEGF.

FIG. 14 shows that Avastin and V_{L}D9 homodimer described herein have different VEGF epitopes.

FIG. 15 shows the expression of V_{L}D9 fused to mouse IgG2c hinge-Fc region which forms a covalently bonded (not drawn) homodimer with a molecular weight of ˜75 kDa. Biolayer interferometry demonstrated that the interaction between VEGF and V_{L}D9 homodimer was not altered by addition of the hinge-Fc domain.

FIGS. 16A-16B show MALLS analysis of V_{L}D9-Fc fusions. (FIG. 16A) Fusion of V_{L}D9-Fc with long native hinge in complex with VEGFA121 (28 kDa). The calculated molecular weight of the complex is 106 kDa (2x V_{L}D9Fc+2x VEGFA). (FIG. 16B) Fusion of V_{L}D9-Fc with short mutant hinge in complex with VEGFA121 (28 kDa). The calculated molecular weight of the complex is 208 kDa (4x V_{L}D9-Fc+4x VEGFA).

FIGS. 17A-17B show proliferation assays in HUVEC cells. (FIG. 17A) Avastin IgG (blue diamonds) and V_{L}D9-Fc native hinge (red squares) were added with VEGFA 165. Avastin decreased proliferation as measured by ³H thymidine incorporation, while V_{L}D9-Fc construct did not. (FIG. 17B) Control IgG (red sqares) and V_{L}D9-Fc short mutant hinge (blue diamonds) did not reduce the proliferation of HUVEC cells. Each data point represents the average of two measurements. This experiment was performed twice with similar results.

FIG. 18 shows the structure of VEGF (blue; central protein) in complex with V_{L}D9 dimer (grey surface representation; middle upper protein) superimposed onto VEGF in complex with VEGFR1 (in magenta, PDB ID 5T89; lower and outermost side protein). No clashes are observed between V_{L}D9 and VEGFR1.

FIG. 19 shows the amino acid sequence of V_{L}D9. CDRs are underlined; putative contact regions are shown in bold (these residues are buried upon VEGF binding).

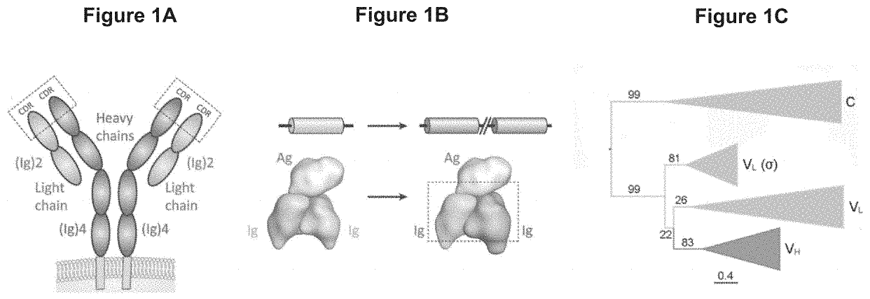

### KEY TO THE SEQUENCE LISTING

SEQ ID NO: 1—Amino acid sequence diversity for immunoglobulin Ig5

SEQ ID NO: 2—Amino acid sequence diversity for CDR1 of immunoglobulin Ig5

SEQ ID NO: 3—Amino acid sequence diversity for CDR2 of immunoglobulin Ig5

SEQ ID NO: 4—Amino acid sequence diversity for CDR3 of immunoglobulin Ig5

SEQ ID NO: 5—Amino acid sequence for immunoglobulin Ig5 cluster family

SEQ ID NO: 6—Amino acid sequence for CDR1 of immunoglobulin Ig5 cluster family

SEQ ID NO: 7—Amino acid sequence for CDR2 of immunoglobulin Ig5 cluster family

SEQ ID NO: 8—Amino acid sequence for CDR3 of immunoglobulin Ig5 cluster family

SEQ ID NO: 9—Amino acid sequence diversity for immunoglobulin Ig12

SEQ ID NO: 10—Amino acid sequence diversity for CDR1 of immunoglobulin Ig12

SEQ ID NO: 11—Amino acid sequence diversity for CDR2 of immunoglobulin Ig12

SEQ ID NO: 12—Amino acid sequence diversity for CDR3 of immunoglobulin Ig12

SEQ ID NO: 13—Amino acid sequence of immunoglobulin Ig12 cluster family

SEQ ID NO: 14—Amino acid sequence for CDR1 of immunoglobulin Ig12 cluster family

SEQ ID NO: 15—Amino acid sequence for CDR2 of immunoglobulin Ig12 cluster family

SEQ ID NO: 16—Amino acid sequence for CDR3 of immunoglobulin Ig12 cluster family

SEQ ID NO: 17—Nucleic acid sequence diversity of oligonucleotide for CDR1

SEQ ID NO: 18—Nucleic acid sequence diversity of oligonucleotide for CDR2

SEQ ID NO: 19—Nucleic acid sequence diversity of oligonucleotide for CDR3

SEQ ID NO: 20—Amino acid sequence for immunoglobulin V_{L}D9

SEQ ID NO: 21—Amino acid sequence for CDR1 of immunoglobulin V_{L}D9

SEQ ID NO: 22—Amino acid sequence for CDR2 of immunoglobulin V_{L}D9

SEQ ID NO: 23—Amino acid sequence for CDR3 of immunoglobulin V_{L}D9

### DETAILED DESCRIPTION

### General Techniques and Definitions

Unless specifically defined otherwise, all technical and scientific terms used herein shall be taken to have the same meaning as commonly understood by one of ordinary skill in the art (e.g., in immunology, immunohistochemistry, protein chemistry, biochemistry and chemistry).

Unless otherwise indicated, the bacterial, genetic, recombinant protein, cell culture, and immunological techniques utilized in the present invention are standard procedures, well known to those skilled in the art. Such techniques are described and explained throughout the literature in sources such as, J. Perbal, A Practical Guide to Molecular Cloning, John Wiley and Sons (1984), J. Sambrook et al., Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al., (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present).

The description and definitions of immunoglobulins, antibodies and fragments thereof herein may be further understood by the discussion in Kabat, 1987 and/or 1991, Bork et al., 1994 and/or Chothia and Lesk, 1987 and/or 1989 or Al-Lazikani et al., 1997.

Those skilled in the art will appreciate that the present disclosure is susceptible to variations and modifications other than those specifically described. It is to be understood that the disclosure includes all such variations and modifications. The disclosure also includes all of the steps, features, compositions and compounds referred to or indicated in this specification, individually or collectively, and any and all combinations or any two or more of said steps or features.

The present disclosure is not to be limited in scope by the specific examples described herein, which are intended for the purpose of exemplification only. Functionally equivalent products, compositions and methods are clearly within the scope of the disclosure, as described herein.

Each feature of any particular aspect or embodiment or example of the present disclosure may be applied mutatis mutandis to any other aspect or embodiment or example of the present disclosure.

Unless specifically defined otherwise, all technical and scientific terms used herein shall be taken to have the same meaning as commonly understood by one of ordinary skill in the art (e.g., in microbiology, bacterial cell culture, molecular genetics, immunology, immunohistochemistry, protein chemistry, biochemistry, and chemistry).

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

As used herein, the singular forms of “a”, “and” and “the” include plural forms of these words, unless the context clearly dictates otherwise.

The term “and/or”, e.g., “X and/or Y” shall be understood to mean either “X and Y” or “X or Y” and shall be taken to provide explicit support for both meanings or for either meaning.

As used herein, the terms “linked”, “attached”, “conjugated”, “bound”, “coupled”, “coated”, “covered”, “adsorbed” or variations thereof are used broadly to refer to any form of covalent or non-covalent association which joins one entity to another for any period of time.

The term “about” as used herein refers to a range of +/−10% of the specified value. For the avoidance of doubt, the term “about” is also to be taken to provide explicit support for the exact number that follows (e.g., the term “about 10” is to be taken to provide explicit support for 10 itself).

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

### Immunoglobulin Homodimer

The term “immunoglobulin” as used shall be taken to mean any protein that contains an immunoglobulin (Ig) domain. Ig domains are commonly known to be, e.g., a tandem series of repeating homology units of roughly 110 amino acid residues in length, which fold independently into a compact globular structure. It is to be understood that all proteins that exhibit this structural motif belong to the immunoglobulin protein superfamily. Ig domains are characterised by an Ig-fold, which has a sandwich-like structure formed by two sheets of antiparallel beta strands. In one example, the Ig domain is an IgV domain. In another example, the Ig domain is an IgC1 domain. In another example, the Ig domain is an IgC2 domain. In yet another example, the Ig domain is an Igl domain.

As used herein, the term “homodimer” shall be understood to refer to a protein complex comprising two identical monomers that bind non-covalently to each other. Accordingly, each monomer of the immunoglobulin homodimers disclosed herein shall be understood to comprise a light chain variable domain (V_{L}). In one example, the V_{L} may be a V_{L} from a kappa (κ) light chain. In another example, the V_{L} may be a V_{L} from a lambda (λ) light chain.

As used herein, the term “light chain variable domain” or “V_{L}” shall be understood to refer to the variable domain of the light chain of an immunoglobulin capable of binding to one or more antigens. The light chain variable domain includes amino acid sequences of complementarity determining regions (CDRs); i.e., CDR1, CDR2, and CDR3, and framework regions (FRs). The three CDRs of the light chain form the antigen binding site. In one example, the present disclosure contemplates any protein that is a homodimer which comprises two identical V_{L}s that specifically and/or selectively bind to one or more antigens.

In the present disclosure, the amino acid positions assigned to CDRs and FRs are numbered using a system for numbering residues in a variable domain of an immunoglobulin in a consistent manner according to a system defined by Kabat (1987 and 1991) and numbered according to the numbering system of Kabat. The person skilled in the art will be readily able to use other numbering systems in the performance of this disclosure, e.g., the hypervariable loop numbering system of Clothia and Lesk (1987) and/or Chothia (1989) and/or Al-Lazikani et al. (1997). For example, CDR2 of a V_{L} is defined at the same position using the numbering system of Kabat or Clothia and Lesk (1987) and/or Chothia (1989).

As used herein, the term “binding specifically” or “specifically binding” shall be understood to mean an immunoglobulin of the present disclosure reacts or associates more frequently, more rapidly, with greater duration and/or with greater affinity with a particular antigen or antigens or cell expressing same than it does with alternative antigens or cells. For example, an immunoglobulin that specifically binds to an antigen may bind to that antigen with greater affinity, avidity, more readily, and/or with greater duration than it binds to other antigens. It is also understood by reading this definition that, for example, an immunoglobulin that specifically binds to a first antigen may or may not specifically bind to a second antigen. As such, “specific binding” does not necessarily require exclusive binding or non-detectable binding of another antigen. Generally, but not necessarily, reference to binding means specific binding, and each term shall be understood to provide explicit support for the other term.

### Plurality

As used herein, the term “plurality” shall be understood to mean two or more. The plurality of immunoglobulin homodimers may comprise a library of immunoglobulin homodimers. The plurality or library may comprise two or more variants of recombinant immunoglobulin homodimers displayed in vitro or in vivo. Displayed variants of immunoglobulin homodimers may be generated from the dimerization of identical variants of recombinant light chain variable domain (V_{L}) monomers. The variants of recombinant immunoglobulin homodimers may be displayed, for example, through a phage display technique. One example of a suitable phage display technique is one that allows any of the identical variants of the V_{L} monomers that are capable of dimerizing, to dimerize. Dimerization may occur in the presence and/or in the absence of an antigen. One suitable means of displaying the V_{L} monomer variants and/or immunoglobulin homodimers is comprises expressing a single variant in a bacteriophage, under conditions that allow multiple copies of the V_{L} monomer to dimerize, e.g. upon contact with an antigen. Thus, any of the methods disclosed herein may comprise contacting the plurality of immunoglobulin homodimers and/or V_{L} monomers with an antigen.

### Light Chain Variable Domain (V_{L}) Monomers

The variants of light chain variable domain (V_{L}) monomers disclosed herein may be generated by any suitable means. For example, the V_{L} monomers may be generated by the modification of V- and J-gene segments through general mutagenesis techniques known in the art. Methods of modifying the sequences encoding V_{L}s from antibodies will be apparent to the skilled artisan and/or described herein. Sequence of V_{L}s for the V- and J-gene segments of known antibodies will be readily obtainable by a person skilled in the art for example, V_{L} sequences of the human V- (O12/O2/DPK9) gene segments and the V_{L} sequence of the human J- (J_{κ}1) gene segments can be readily obtained from publically available databases. As used herein, the term “modified” in the context of a V_{L} means that the V- and J-segments sequence of the V_{L} is changed compared to a parent (or unmodified) V_{L}.

Polynucleotides (e.g., DNA) encoding the V- and J-gene segments of the V_{L} domain can be isolated using standard methods in the art. For example, primers can be designed to anneal to conserved regions of the V- and J-gene segments within the light chain variable domain that flank the region of interest, and those primers are then used to amplify the intervening nucleic acid, e.g., by PCR. Suitable methods and/or primers are known in the art and/or described, for example, in Borrebaeck (ed), 1995 and/or Froyen et al. 1995.

Following amplification and isolation, the DNA can be modified to include codons encoding variant amino acids at the requisite locations by any of a variety of methods known in the art. In one example, site-directed mutagenesis can be performed at specific positions within one or more hypervariable CDRs (e.g., positions 28, 30-32, 50-51, 53, 91-94, 96; numbering according to Kabat) to produce variants of immunoglobulin monomers as disclosed herein. This technique is known in the art (see for example, Carter et al. 1985; or Ho et al. 1989). Briefly, in carrying out site-directed mutagenesis of DNA, the starting DNA is altered by first hybridizing an oligonucleotide encoding the desired mutation to a single strand of such starting DNA. After hybridization, a DNA polymerase is used to synthesize an entire second strand, using the hybridized oligonucleotide as a primer, and using the single strand of the starting DNA as a template. Thus, the oligonucleotide encoding the desired mutation is incorporated in the resulting double-stranded DNA. Site-directed mutagenesis may be carried out within the gene expressing the protein to be mutagenized in an expression plasmid and the resulting plasmid may be sequenced to confirm the introduction of the desired negatively charged amino acid replacement mutations. Site-directed protocols and formats include commercially available kits, e.g. QuikChange@ Multi Site-Directed Mutagenesis Kit (Stratagene, La Jolla, Calif.). In another example, mutagenesis may be introduced using PCR mutagenesis. In yet another example, mutagenesis may be introduced using cassette mutagenesis.

The modified V- and J-segments can be randomly recombined by methods known in the art to create continuous exons that encode the whole recombinant V_{L} region resulting in variants of recombinant proteins possessing combinatorial diversity. In one example, variants of recombinant proteins may be constructed by splice overlap extension PCR with synthetic oligonucleotides as known in the art. In another example, variants of recombinant proteins may be constructed by restriction fragment manipulation. General guidance can be found in Sambrook et al. 1989; and/or Ausubel et al. 1993. The V_{L} monomer described herein may comprise modified V- and J-gene segments.

### Phage Display

The V_{L} monomers disclosed herein may be capable of dimerizing to form immunoglobulin homodimers. Thus, the present disclosure provides a method for generating a plurality of V_{L} monomers under conditions that allow for V_{L} monomers that are capable of dimerizing, to dimerize. In one example as disclosed herein, the method may comprise the use of a phage display technique. The phage display technique may comprise expressing multiple copies of a V_{L} monomer joined to a filamentous phage protein via a linker protein. The linker protein may comprise a sufficient number of amino acids to allow sufficient conformational flexibility to enable V_{L} monomer dimerization.

It will be understood that not all V_{L} monomers in the plurality of V_{L} monomers (e.g., not all V_{L} monomers in the library) may be capable of dimerizing to form an immunoglobulin homodimer. However, amongst the entire plurality (or library), it is anticipated that one or more V_{L} monomers will be capable of dimerizing, either in the absence or the presence of an antigen. Those V_{L} monomers that are capable of dimerizing can be identified using the screening methods disclosed herein.

As used herein, the terms “phage display” and “bacteriophage display” are well known in the art and shall be understood to mean a laboratory technique by which variant polypeptides and/or proteins are displayed as fusion proteins to a coat protein on the surface of a phage. In one example, the recombinant V_{L} monomers may be displayed using a phagemid display format as exogenous polypeptides and/or proteins on the surface of a phage. In another example, the recombinant V_{L} monomers may be displayed on the surface of a phage using phage display format.

Accordingly, nucleic acids of the sequences encoding for the recombinant V_{L} monomers described herein can be amplified, isolated and cloned into any suitable phage display vector known in the art. The term “phage display vector” used herein shall be understood to refer to a phage vector that could be used to display exogenous V_{L} monomer proteins on the surface of a bacteriophage. In one example, the phage display vector may be a phagemid vector. The term “phagemid vector” as used herein shall be understood to refer to a geneIII-expressing plasmid that contains f-phage packaging signals, an f1-phage origin of replication from an f1-phage, appropriate multiple cloning sites designed to genetically fuse a polypeptide and/or protein at the N-terminus of a pIII C-terminal domain as a fusion protein, and a suitable selection marker. In one example disclosed herein, the phagemid vector may be pHEN1. In another example, the phagemid vector may include pComb3, pComb8 or pSEX. In another example, the phage display vector is a phage vector in which the phage vector may include: fUSE5, fAFF1, fd-CAT1 or fdtetDOG.

Nucleic acids encoding for the V_{L} monomer polypeptides and/or proteins, upon cloning into a phagemid vector, can be fused to the nucleic acids of the sequence encoding for the pIII phage coat protein resulting in a V_{L} monomer-geneIII construct. All geneIII proteins expressed from the phagemid vector may be fusion proteins. In another example, the nucleic acids of the sequence encoding for the V_{L} monomer polypeptides and/or proteins upon cloning into a phagemid vector may be fused to a pVIII phage coat protein.

The phagemid vector comprising the V_{L} monomer-geneIII fusion construct can be transfected into any suitable bacterial host cell known in the art that is compatible with the phagemid vector. In one example as disclosed herein, the host cell is E. coli TG1 bacteria. In another example, the E. coli host cell may include: SS320, ER2738, or XL1-Blue. It would be understood in the art that no V_{L} monomer proteins may be expressed at this stage if the phagemid lacks structural and non-structural gene products required for generating a complete phage.

The method as disclosed herein, also provides for the phagemid-containing E. coli host cells to be infected with a bacteriophage. As used herein, the term “bacteriophage” or “phage” shall be understood to refer to mean any group or type of viruses that infects bacteria in order to replicate. In one example as disclosed herein, the bacteriophage may be an M13 filamentous bacteriophage that infects bacterium Escherichia coli. In another example, the filamentous bacteriophage may include: an f1 phage, an fd phage or a Pfl phage. In yet another example, the bacteriophage may include: a T4 phage, a T7 phage of a lambda phage.

As used herein, the term bacteriophage shall also be understood to include a helper phage. The term “helper phage” as used herein shall be understood to refer to a variant of bacteriophage that contains a slightly defective origin of replication and serves to supply in trans, all the structural proteins required in order to enable the packaging of the recombinant phagemid DNA containing V_{L} monomer DNA, and assembly of complete filamentous M13 phage particles. In one example as disclosed herein, the helper phage may be M13KO7. In another example, the helper phage may be VCSM13. Generated M13 phage particles disclosed herein may express and display multiple copies of V_{L} monomer-geneIII fusion polypeptides and/or proteins on its surface. In one example as disclosed herein, the V_{L} monomer-geneIII fusion polypeptides and/or proteins may be expressed on the surface of the phage particle as an antibody fragment.

The number of copies of V_{L} monomer-geneIII fusion polypeptides and/or proteins displayed on the surface of phage particles may be between about 2-10 copies. For example, the number of copies of V_{L} monomer-geneIII fusion polypeptides and/or proteins displayed on the surface of phage particles may be between 2-10 copies, such as between 2-9 copies, such as between 2-8 copies, such as between 2-7 copies, such as between 2-6 copies, such as between 2-5 copies, such as between 2-4 copies and such as between 2-3 copies. In one example, the number of copies of V_{L} monomer-geneIII fusion polypeptides and/or proteins displayed on the surface of phage particles may be between 2-8 copies such as between 2-5 copies and such as between 3-5 copies. The above measurements may apply to each individual phage particle or may apply to the average of all phage particles used in any particular instance.

The proximity of a plurality of V_{L} monomer copies displayed on the surface of the phage particles to one another as described herein, allows for identical V_{L} monomers capable of dimerizing, to dimerize, thereby resulting in the formation of immunoglobulin homodimers.

The phage particles expressing antibody fragments comprising the immunoglobulin homodimers on its surface generated herein, may be assembled to construct a phage display library.

### Screening Methods: Affinity Screening

The plurality (or library) of immunoglobulin homodimers and/or V_{L} monomers described herein may be screened to select for immunoglobulin homodimers with strong binding towards a target.

Methods for screening display libraries to a target are known in the art. In one example, a display library of the present disclosure is screened using bio-panning procedure. As used herein, the term “bio-panning” shall be understood to refer to an affinity selection technique which selects for peptides that bind to a given target. The biopanning procedure would be known to the skilled person in the art and generally involves five steps:

i) preparation of a phage display library comprising a plurality of phage particles displaying immunoglobulin homodimers and/or V_{L} monomers on its surface,

ii) exposure and incubation of the phage particles to a target molecule immobilized on a solid support,

iii) removal of non-specific phage particles that express immunoglobulin homodimers and/or V_{L} monomers that do not bind to the target through washing,

iv) recovery of phage particles expressing immunoglobulin homodimers and/or V_{L} monomers that bind to the target by elution or direct bacterial infection and amplification of the recovered phage, and

v) repeat steps i) to iv) above for two to four rounds.

The term “solid support” used herein shall be understood to refer to the surface on which the target molecule is immobilized which may include: microtiter plate wells, PVDF membrane, column matrix or immunotubes, magnetic beads or on whole cells.

From the steps above, the bio-panning screening procedure can be repeated several times until a population of phage particles that express immunoglobulin homodimers with the best binding affinity to the target is identified. Binding affinity of immunoglobulin homodimers to the target may be assessed using any laboratory technique known in the art. In one example as disclosed herein, binding affinity may be assessed using enzyme-linked immunosorbent assay. In another example, binding affinity may be assessed using: fluorometric microvolume assay technology (FMAT) or chromophore-assisted laser inactivation (CALI).

As used herein, the term “target” shall be understood to refer to an antigen. As used herein, the term “antigen” shall be understood to mean any composition of matter against which an immunoglobulin response (e.g., an antibody response) could potentially be raised. Exemplary antigens include proteins, peptides, polypeptides, carbohydrates, phosphate groups, phosphor-peptides or polypeptides, glyscosylated peptides or peptides, etc.

It will also be readily understood in the art that the antigen may comprise a protein that is a single protein. In addition, it will also be readily understood in the art that the antigen may comprise a protein that is composed of two or more subunit proteins bound together. For example, many proteins exist as multimeric composition. The term “antigen” as used herein is also intended to refer to a multimeric protein. The component parts of the multimeric protein may be the same or different. Thus, the multimeric protein may be, for example, a homodimer or a heterodimer, or a homotetramer or a heterotetramer, etc. The present disclosure demonstrates that the immunoglobulin homodimers disclosed herein are capable of binding with high affinity to symmetrical antigens. Symmetrical proteins are often composed of multimers. The present disclosure also demonstrates that the immunoglobulin homodimers disclosed herein are capable of binding to a symmetrical antigen without interfering with (e.g., without inhibiting) the normal activity of that antigen.

It will also be understood that the antigen may comprise proteins having various symmetrical forms. The term “symmetry” or “symmetrical” as used herein shall be understood to refer to the shape of the antigen molecule when assessed along an axis or sagittal plane that divides the molecule into equivalent left and right halves. It would be further understood in the art that symmetry of a molecule may be assessed when the left and right halves of the molecule are rotated, inverted or reflected along the axis or sagittal plane. An antigen may be an asymmetric antigen in which the left and right halves of the protein molecule are not identical when assessed along an axis or sagittal plane. In one example as used herein, the asymmetric antigen may be hen egg-white lysozyme (HEL). An antigen may be a symmetrical antigen if the left and right halves of the antigen are identical when assessed along an axis or sagittal plane. In one example as used herein, a symmetrical antigen may be vascular endothelial growth factor (VEGF). Other classes of symmetrical antigens are contemplated, including physiological signalling molecules.

As disclosed herein, immunoglobulin homodimers that bind with strong affinity to an antigen target result in the formation of an immunoglobulin homodimer-antigen complex. As used herein, the term “immunoglobulin homodimer-antigen complex” shall be understood to mean a complex formed by the binding of an immunoglobulin homodimer to an antigen through chemical interactions, and bonding is essentially non-covalent. Ionic interactions, hydrogen bonds, van der Waals forces, and hydrophobic interactions may be involved depending on the interaction sites.

As used herein, the term “affinity” shall be understood to refer to the strength of interaction between the binding sites of the immunoglobulin homodimers disclosed herein with the antigen. In one example described herein, strength of interaction may be determined by measuring the strength of binding reaction through affinity constants which are well known in the art. In one example, strength of binding between the immunoglobulin homodimers described herein and the antigen may be determined by measuring the association constant (Kₐ) and/or the association constant dissociation constant (K_{d}) and/or the equilibrium binding constant (K_{D}). The method described herein provides that affinity may be measured using any suitable techniques, including any suitable optical analytical techniques for measuring biomolecular interactions known in the art. In one example, optical analytical techniques may include: Bio-Layer

Interferometry. In another example, the optical analytical technique may include: dual polarisation interferometry, static light scattering, dynamic light scattering, surface plasmon resonance, fluorescence polarisation/anisotropy, fluorescence correlation spectroscopy or nuclear magnetic resonance.

As disclosed herein, the affinity of immunoglobulin homodimers for the antigen may be determined from the values obtained for equilibrium binding constant (K_{D}). The values for equilibrium binding constant (K_{D}) may be micromolar or nanomolar. In one example, the affinity of immunoglobulin homodimers to the antigen may be in the range of about 1 nM to about 1 μM. For example, the affinity of immunoglobulin homodimers to the antigen may be in the range of about 10 nM to about 500 nM, such as about 10 nM to about 200 nM, such as about 10nM to about 100 nM. The methods disclosed herein may comprise selecting an immunoglobulin homodimer having an affinity for an antigen within any of these ranges.

In another example, the affinity of immunoglobulin homodimers to the antigen may be less than about 100 μM. In another example, the affinity of immunoglobulin homodimers to the antigen may be less than about 50 μM, such as less than about 10 μM, such as less than about 1 μM, such as less than about 900 nM, such as less than about 800 nM, such as less than about 700 nM, such as less than about 600 nM, such as less than about 500 nM, such as less than about 400 nM, such as less than about 300 nM, such as less than about 200 nM, such as less than about 100 nM, such as less than about 90 nM, such as less than about 80 nM, such as less than about 70 nM, such as less than about 60 nM, such as less than about 50 nM, such as less than about 40 nM, such as less than about 30 nM, such as less than about 20 nM, such as less than about 10 nM, such as less than about 5 nM. Preferably, the affinity of immunoglobulin homodimers to the antigen may be less than about 100 nM. More preferably, the affinity of immunoglobulin homodimers to the antigen may be less than about 10 nM. The methods disclosed herein may comprise selecting an immunoglobulin homodimer having an affinity for an antigen within any of these ranges.

In addition, affinity of immunoglobulin homodimers for the antigen may also be determined from the values obtained for the buried surface area of the immunoglobulin homodimers and/or the buried surface area of the immunoglobulin homodimer-antigen complex. The term “buried surface area” as used herein, shall be understood to refer to the total surface area calculated on atomic coordinates (Å²), of the immunoglobulin homodimer at the point of contact with the antigen. It shall be understood in the art that the greater the surface area covered between immunoglobulin homodimer and the antigen, the greater the affinity of the immunoglobulin homodimer has towards the antigen. In one example, the buried surface of the immunoglobulin homodimers described herein with an antigen may be in the range of about 650 Å² to about 880 Å². In another example the buried surface of the immunoglobulin homodimers described herein with an antigen may be in the range of about 660 Å² to about 870 Å². In another example the buried surface of the immunoglobulin homodimers described herein with an antigen may be in the range of about 670 Å² to about 860 Å². In another example the buried surface of the immunoglobulin homodimers described herein with an antigen may be in the range of about 680 Å² to about 850 Å². In yet another example the buried surface of the immunoglobulin homodimers described herein with an antigen may be in the range of about 690 Å² to about 830 Å². The methods disclosed herein may comprise selecting an immunoglobulin homodimer having a buried surface area within any of these ranges.

The buried surface of the immunoglobulin homodimers described herein with an antigen may be more than about 500 Å², such as more than about 550 Å², such as more than about 600 Å², such as more than about 650 Å², such as more than about 660 Å², such as more than about 670 Å², such as more than about 700 Å², such as more than about 750 Å², such as more than about 800 Å², such as more than about 850 Å². The methods disclosed herein may comprise selecting an immunoglobulin homodimer having a buried surface area within any of these ranges.

The immunoglobulin homodimers described herein may bind to any particular parts of the antigen to form the immunoglobulin homodimer-antigen complex. In one example, the immunoglobulin homodimers described herein may bind to the antigen on a planar surface of the antigen. In another example, the immunoglobulin homodimers described herein may bind to the antigen within an active site cleft on the surface of the antigen. In yet another example, the immunoglobulin homodimers described herein may bind to the antigen in a region not within an active site cleft on the surface of the antigen.

Particular amino acids amongst the immunoglobulin homodimers disclosed herein will form the contact points to the antigen. In one example, the amino acid tyrosine may be abundant in antigen-binding sites as coincidental consequence of biases that exist in the base composition of antibody genes or alternatively, because tyrosine is particularly well suited for molecular recognition (as described by Zemlin et al. (2003)). In one example of the present disclosure, the immunoglobulin homodimers may comprise identical immunoglobulin monomers with independent binding sites containing tyrosine residues at specific positions that enable the binding site to adopt different conformations to complement binding to an asymmetric or symmetric antigen. In one example as described herein, the immunoglobulin homodimers may comprise one or more tyrosine residues at positions 53 and/or 95 according to the numbering system of Kabat.

Screening methods: dimerization screening of immunoglobulin homodimer—antigen complex The immunoglobulin -antigen complexes described herein may be screened for immunoglobulin homodimers using any analytical techniques known in the art that can be used to determine the stoichiometric ratio of the V_{L} monomer to antigen molecule. As used herein, the term “stoichiometric ratio” or “stoichiometry” shall be understood to refer to the relative numbers of V_{L} monomers and antigen molecules in the immunoglobulin-antigen complex. In one example, the stoichiometric ratio of V_{L} monomers to antigen may be 2:1. It will be appreciated that, if the antigen is a multimeric molecule, the entire multimeric antigen should be considered to be one molecule (rather than any subunit or monomer thereof) when determining the stoichiometric ratio as described herein. A stoichiometric ratio of 2:1 shall be understood to mean that two V_{L} monomers (together forming one immunoglobulin homodimer) bind to a single antigen. As described herein, it shall be understood that an immunoglobulin homodimer-antigen complex may be in a stoichiometric ratio of 2:1.

It will also be understood that, if two immunoglobulin homodimers bind to a single antigen, the stoichiometric ratio will be 4:1. The methods disclosed herein may also comprise selecting an immunoglobulin homodimer when the stoichiometric ratio of immunoglobulin monomer to antigen in the immunoglobulin-antigen complex is about 4:1.

The stoichiometric ratio of the immunoglobulin homodimer-antigen complexes may be determined using any suitable means. For example, a method that measures the molecular weight of proteins may be used. The molecular weight of the immunoglobulin homodimer-antigen complex may be determined using a combination of analytical techniques which comprises any chromatography methods known in the art to separate protein molecules according to their molecular weight, together with any spectroscopic analytical techniques involving light scattering known in the art to determine the concentration of the protein molecules eluted. In one example as described herein, the spectroscopic analytical technique involving light scattering is multi-angled laser light scattering (SEC-MALLS). It shall be further understood in the art that the concentration of molecules eluted may be depicted as elution peaks on paper and/or on an electronic display from which molecular weights of the molecules may be calculated using Debye fitting of the elution peaks. In another example, stoichiometric ratio of the immunoglobulin homodimer-antigen complexes may be determined through crystallization of the immunoglobulin homodimer-antigen complex.

In one example as described herein, the immunoglobulin-homodimer antigen complexes may be provided in the form of a mixture to the size-exclusion chromatography column It will be understood that the mixture may contain a plurality of different protein molecules which may comprise two or more of: the immunoglobulin homodimer-antigen complex, the immunoglobulin homodimer molecule unbound to the antigen and/or the antigen molecule unbound to the immunoglobulin homodimer. The protein molecules may be eluted out of the size-exclusion chromatography column and their molecular weights calculated. The calculated molecular weights of two or more of the molecules may be compared to determine the stoichiometric ratio of immunoglobulin monomer to antigen in the immunoglobulin homodimer-antigen complex.

Alternatively, a mixture comprising a plurality of immunoglobulin homodimer-antigen complex may be optionally pre-prepared prior to contacting the mixture with the size-exclusion chromatography column In one example as disclosed herein, pre-preparation of an immunoglobulin-antigen complex may comprise incubating an equimolar amount of purified immunoglobulin homodimer with the antigen in a suitable buffer. Any suitable buffer may be used. In one example as disclosed herein, the buffer used for the incubation is phosphate buffered saline (PBS).

The purified immunoglobulin and the antigen may be incubated at room temperature. In one example as disclosed herein, the temperature may be in the range of about 18° C. to about 28° C., such as about 18 ° C. to about 26° C., such as about 20° C. to about 26° C. Preferably, the purified immunoglobulin and the antigen may be incubated to a temperature in the range of about 20° C. to about 25° C.

In one example, the incubation may be performed for up to 15 minutes, up to or 30 minutes, or up to 45 minutes, or up to 60 minutes, or up to 75 minutes, or up to 90 minutes, or up to 105 minutes or up to 120 minutes. Preferably, incubation may be performed for up to 60 minutes. Alternatively, incubation may be performed for at least about 30 minutes, or at least about 45 minutes, or for at least about 60 minutes, or for at least about 75 minutes, or for at least about 90 minutes. Preferably, the incubation may be performed for at least about 60 minutes.

### Physiological Signalling Molecule

As disclosed herein, the term “physiological signalling molecule” shall be understood to mean any molecule that transmits information between the cells of multicellular organisms. All physiological signalling molecules act as ligands that bind to receptors expressed by their target cells, there is considerable variation in the structure and function of the different types of molecules that serve as signal transmitters. In one example, the physiological signalling molecule disclosed herein comprises a peptide or a protein. In another example, the physiological signalling molecule disclosed herein comprises a lipid i.e. eicosanoids. In another example, the physiological signalling molecule disclosed herein comprises a hormone. In another example, the physiological signalling molecule disclosed herein comprises a neurotransmitter. In yet another example, the physiological signalling molecule disclosed herein comprises a cytokine. In yet another example, the physiological signalling molecule disclosed herein comprises a steroid.

### Polynucleotide

As used herein, the term “polynucleotide” shall be understood to include DNA, RNA, or a combination of these, single or double stranded, in the sense or antisense orientation or a combination of both, dsRNA or otherwise. The polynucleotide may include one or more chemical modifications which increase its stability and/or efficacy. The polynucleotide may be an isolated polynucleotide. The term “isolated polynucleotide” refers to a polynucleotide which is at least partially separated from the polynucleotide sequences with which it is associated or linked in its native state. Preferably, the isolated polynucleotide is at least 60% free, preferably at least 75% free, and most preferably at least 90% free from other components with which they are naturally associated. Furthermore, the term “polynucleotide” is used interchangeably herein with the term “nucleic acid”.

### Proteins

As used herein, the term “protein” shall be understood to include a single polypeptide, i.e., a series of contiguous amino acids linked by peptide bonds or a series of polypeptides covalently or non-covalently linked to one another (i.e., a polypeptide complex). For example, the series of polypeptides can be covalently linked using a suitable chemical or a disulphide bond. Examples of non-covalent bonds include hydrogen bonds, ionic bonds, Van der Waals forces, and hydrophobic interactions. A non-covalent bond contemplated by the present disclosure is the interaction between a V_{L} and a V_{L}, to form a homodimer.

### Immunoglobulin Homodimer Modifications

The immunoglobulin homodimers identified and/or provided and/or selected by any of the methods disclosed herein may be further modified to improve their stability. Thus, the methods disclosed herein may further comprise a step of binding the immunoglobulin homodimer identified and/or provided and/or selected by any of the methods disclosed herein to a stability-enhancing agent. Suitable stability-enhancing agents are known in the art. In one example, the methods disclosed herein may further comprise a step of binding the immunoglobulin homodimer to an Fc portion. The immunoglobulin homodimer may be bound to an Fc portion via a linker peptide. One or more immunoglobulin homodimers may be bound to an Fc portion. Where more than one immunoglobulin homodimer is bound to an Fc portion, those immunoglobulin homodimers may be the same or may be different. When those immunoglobulin homodimers are different, the resulting molecule may thus be able to bind specifically to two or more different antigens. Thus, the methods disclosed herein may comprise providing a bispecific antibody-like molecule.

The immunoglobulin homodimers identified and/or provided and/or selected by any of the methods disclosed herein may be humanized. Suitable methods for humanizing variable domain sequences originally derived from a non-human animal are well known in the art. Any of those methods may be incorporated into the methods disclosed herein to produce a humanized immunoglobulin homodimers that is particularly suitable for administration to a human subject.

The immunoglobulin homodimers identified and/or provided and/or selected by any of the methods disclosed herein may be further modified by binding them to a detectable label. Any detectable label known to be suitable for labelling an antibody may be used. Thus, the methods disclosed herein may further comprise a step of binding the immunoglobulin homodimer identified and/or provided and/or selected by any of the methods disclosed herein to a detectable label. This further enhances the utility of the immunoglobulin homodimers in many of their potential applications, including in research, diagnostic and/or therapeutic applications.

### EXAMPLES

### Example 1

### Phage Library Design and Construction

The inventors set out to design and construct a phage library that would allow for the selection and characterisation of antigen receptors comprising immunoglobulin homodimers.

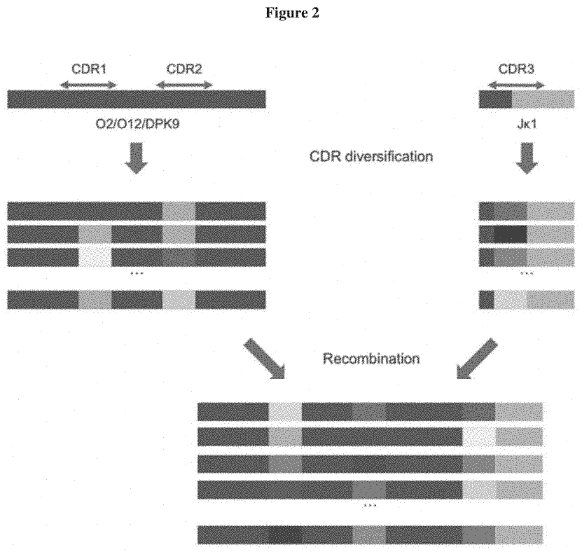

The inventors structurally reconstructed a single-domain immunoglobulin homodimer scaffold by utilizing V- and J-segments of the human antibody light chain family. The genes encoding V-(O12/O2/DPK9) and J-gene segments (Jκ1) of the human light chain were amplified by PCR and cloned into the phage display vector pHEN1 according to the methods as described by Hoogenboom et al. (1991). V-segment diversity was introduced at hypervariable CDR1 and CDR2 positions 28, 30-32, 50-51, 53, 91-94 and 96; (numbering according to the system of Kabat) using Kunkel mutagenesis according to the methods as described in Kunkel (1985) while J-segment diversity was introduced by PCR using degenerate TRIM oligonucleotides (Table 1) using the methods as described in Kunkel (1985) and Rouet et al. (2012). Combinatorial diversity was generated through recombination of the V- and J-segments using splice overlap extension PCR (FIG. 2), followed by cloning into display vector pHEN1 and transformation into E. coli TG1 bacteria. This resulted in a library of phage constructs with each bacteriophage displaying multiple copies of the receptor on the tip of its filamentous surface as described in Lee et al. (2007). Those multiple copies of each receptor were found to be able to dimerise, for example, upon binding an antigen.

TABLE 1
TRIM oligonucleotides used to introduce J-segment diversity for
phage library construction.

| | |
|---|---|
| CDR1 | 5′-CATCACTTGCCGGGCAAGTCAGKMTATTKMTKMTKMTTTAAATTGGTATCAGCAGAA |
| | AC-3′ |
| CDR2 | 5′-CAGGGAAAGCCCCTAAGCTCCTGATCTATKMTKMTTCCKMTTTGCAAAGTGGGGTCC |
| | CATCAAG-3′ |
| CDR3 | 5′-ACTTACTACTGTCAACAGXxxXxxXxxXxxCCTXxxACGTTCGGCCAAGGGACCAAG-3′ |

Diversified positions in bold. CDR1 and CDR2 oligonucleotides were
reverse-complemented and phosphorylated before use in Kunkel mutagenesis.
KMT encoded amino acids (codons):

| | |
|---|---|
| 25% | Y(TAT) |
| 25% | S(TCT) |
| 25% | A(GCT) |
| 25% | D(GAT) |

Xxx encoded amino acids (TRIM codons):

| | |
|---|---|
| 19.7% | Y(TAC) |
| 15.2% | S(TCT) |
| 6.6% | A(GCT) |
| 6.6% | D(GAC) |
| 16.7% | G(GGT) |
| 3.9% | I(ATC), L(CTG), P(CCG), R(CGT), T(ACT), V(GTT) |
| 1.6% | E(GAA), F(TTC), H(CAT), K(AAA), M(ATG), N(AAC), Q(CAG), |
| | W(TGG) |

### Example 2

### Selection of Antigen Binders

The inventors next sought to select single-domain immunoglobulin phage clones that could selectively bind to an asymmetric antigen (exemplified here by using hen egg-white lysozyme (HEL)) and/or a symmetric antigen (exemplified by vascular endothelial growth factor (VEGF)).

The inventors panned the phage library against hen egg-white lysozyme (HEL) (Sigma- Aldrich), which had been biotinylated using NHS-PEG4-biotin (Thermo-Fisher) according to manufacturer's instructions. For rounds 1 and 3 of the selection, the HEL antigen was captured using neutravidin (Thermo-Fisher) coated wells of a MaxiSorp Immunoplate (Nunc). For rounds 2 and 4 of the selection, magnetic streptavidin beads (Invitrogen) were used as an alternative means of capture to prevent the selection of binder against the capture reagents. After four rounds of selection, binders were identified by soluble fragment ELISA according to the methods as described in Rouet et al. (2012). This selection step allowed the identification of antigen specific phage clones.

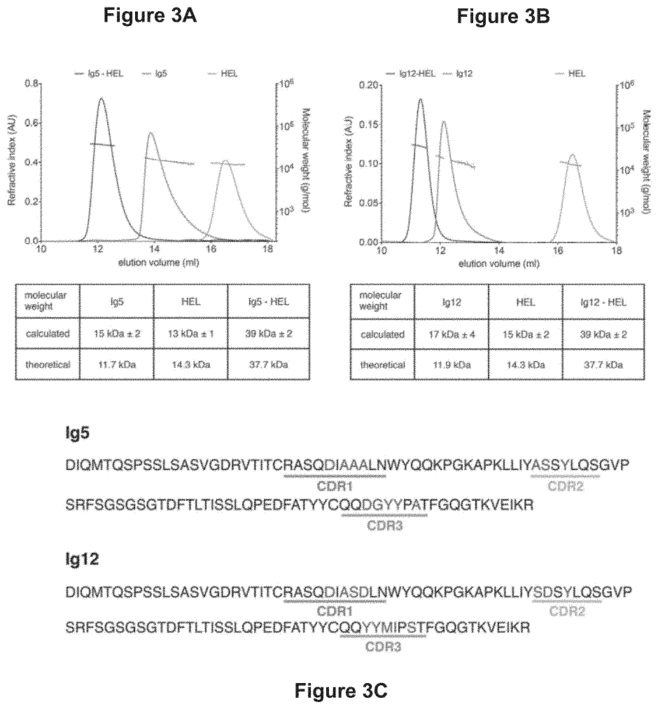

From the antigen selection process, the inventors identified twelve unique recombinant clones, all of which were sequenced. From the sequencing results, the inventors found that the clones clustered into two families based on their recombinant sequence signature. These two family clusters were designated by the inventors as Ig5 and Ig12 respectively (FIG. 3C). A single representative was chosen from each family for protein expression and purification.

The same methods described above were used to select and identify recombinant clones that would bind to an exemplary symmetric antigen target, VEGF. From the antigen selection process and sequencing, the inventors identified one particular clone that binds to VEGF for further investigation; designated as ‘V_{L}D9’. The amino acid sequence of V_{L}D9 is described herein as SEQ ID NO: 20. The CDR1, CDR2 and CDR3 sequences of V_{L}D9 are described herein as SEQ ID NOs: 21, 22 and 23, respectively.

### Example 3

### Protein Expression and Purification

The inventors then sought to express and purify the protein of each of the representative clones from the Ig5 and Ig12 family clusters, respectively.

Regions encoding the single-domain immunoglobulin homodimer of the clones were amplified by PCR from the phagemid vectors and subsequently cloned into the periplasmic expression vector pET12a (Novagen). This resulted in clone constructs for Ig5 and Ig12 respectively, that were transformed into E. coli BL21-Gold (DE3) (Agilent). Protein expression was induced in mid-exponential cultures by addition of 1 mM isopropyl β-D-1- thiogalactopyranoside (Gold Biotechnology), and the single-domain immunoglobulin homodimers were purified from filtered supernatant using Protein L affinity resin (Genscript) according to manufacturer's instructions. Generally the same protocol was followed to express the V_{L}D9 clone.

### Example 4

### Determination of Binding Stoichiometry

The inventors then sought to determine the binding stoichiometry of the expressed and purified single-domain immunoglobulin homodimer from the Ig5 and Ig12 cluster families, respectively.

The inventors performed a dimerization screening step comprising size exclusion chromatography coupled with multi-angle laser light scattering (SECMALLS) according to the methods described in Ye (2006) to measure the molecular weight of the immunoglobulin-antigen complex. Additionally, the inventors used the same technique to measure the molecular weights of the immunoglobulin alone and the antigen alone. The inventors then compared the measured molecular weights of the immunoglobulin-antigen complex, immunoglobulin alone and antigen alone respectively. The inventors thereby determined the immunoglobulin to antigen stoichiometry from differences in the measured molecular weights.

Immunoglobulin-antigen complexes for Ig5 and Ig12 respectively were generated by incubating equimolar amounts of purified immunoglobulin homodimers Ig5 and Ig12 respectively, with HEL antigen for one hour at room temperature.

Analyses of the immunoglobulin-antigen complexes were performed using a Superdex 75 10/300 GL size exclusion chromatography column (GE Healthcare) using an Äkta Purifier HPLC system (GE Healthcare) at a flow rate of 0.5 mL/min in phosphate buffered saline (PBS). Column eluates containing the compounds immunoglobulin alone, antigen alone and the immunoglobulin-antigen complex for Ig5 (Ig5-HEL) and Ig12 (Ig12-HEL) respectively, were analyzed using a miniDAWN Tristar laser light scattering photometer and an Optilab DSP interferometric refractometer (Wyatt Technology Corporation). Molecular weights of the compounds were calculated using Debye fitting of elution peaks which are well known in the art.

From the measured molecular weights illustrated in FIG. 3, the inventors found that the stoichiometry of immunoglobulin-antigen complexes Ig5-HEL and Ig12-HEL respectively were 2:1. The measured molecular weights for both Ig5-HEL and Ig12-HEL complexes were consistent with two Ig domains bound to a single HEL molecule in the immunoglobulin-antigen complex.

### Example 5

### Affinity Measurements

After confirming that the stoichiometry of the immunoglobulin-antigen complex for Ig5 and Ig12 respectively was 2:1 (i.e., Ig5₂-HEL, Ig12₂-HEL), the inventors sought to measure the affinity of homodimers Ig5 and Ig12 respectively to the asymmetric HEL antigen.

Binding kinetics of immunoglobulin-antigen complexes Ig52-HEL and Ig122-HEL respectively, were analyzed using Bio-Layer Interferometry (ForteBio). Hen egg white lysozyme antigen was captured on streptavidin biosensors using biotinylated HyHEL-10 (Ig5) and HyHEL-5 (Ig12) antibodies in Fab format (expressed using the Expi-293 expression system (Thermo Fisher Scientific)). Affinity of soluble purified Ig5 and Ig12 to HEL was performed using serial dilutions of the Ig component on a ForteBio BLItz instrument, using global fitting and the BLItz Pro 1.1.

Binding kinetics measured were the association constant (Kₐ), dissociation constant (K_{d}) and the equilibrium binding constant (K_{D}). From the measured biding kinetics obtained, the inventors demonstrated that the single-domain immunoglobulin homodimers of Ig5 and Ig12 respectively had binding affinities in the nanomolar range. These binding affinities were orders of magnitude higher than binding affinities previously reported for Bence-Jones proteins. Preston—can you include a reference here that discusses the Bence Jones affinities.

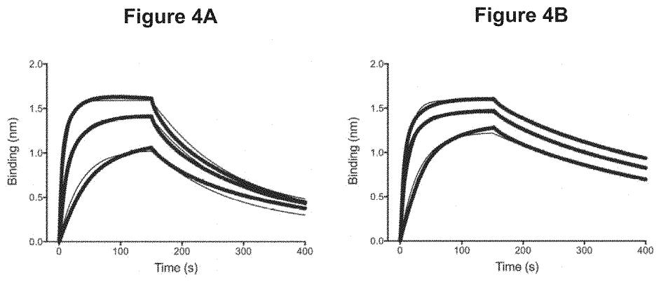

From the measured binding kinetics illustrated in FIG. 4, the inventors were able to assess if the single-domain antibody homodimers are as effective in interacting with a symmetric or asymmetric antigen in a manner that is typical of bona fide immunoglobulins or previously known single-domain antibodies (e.g., Bence-Jones proteins).

Immunoglobulin homodimer Ig5 was tested at concentrations of 2000 nM, 1000 nM and 500 nM concentrations and the affinity measurements obtained were kₐ=4.3·104 M⁻¹s⁻¹; k_{d}=5.7.10⁻³ s⁻¹ and K_{D}=1.3·10⁻⁷ M).

Immunoglobulin homodimer Ig12 was tested at concentrations of 1000 nM, 750 nM and 375 nM concentrations and the affinity measurements obtained were kₐ=8.4·10⁴ M⁻¹s⁻¹; k_{d}=2.5·10⁻³ s⁻¹; K_{D}=3.0·10⁻⁸M.

Results from these binding studies using biolayer interferometry revealed affinities in the nanomolar range, with equilibrium binding constants (KD) for HEL binding of 130 nM and 30 nM for Ig5 and Ig12, respectively (FIG. 4). The Ig5 and Ig12 structures disclosed here are characterized by affinities that are orders of magnitude higher (nanomolar) than previously studied single domain antibodies. For example, the Ig5 and Ig12 affinities are surprisingly in stark contrast with the estimated low affinity (micromolar) of the Bence Jones protein MCG (Edmundson et al. 1993 and Edmundson and Manion, 1998).

Generally the same method described above was used by the inventors to measure the affinity of V_{L}D9 homodimer to VEGF antigen. The affinity measurement obtained for V_{L}D9 to VEGF was a k_{d} of less than 10 nM.

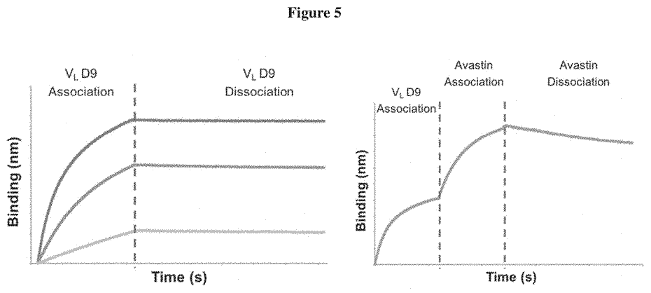

The inventors further used Biolayer interferometry to compare V_{L}D9 binding to VEGF with the binding of the monoclonal antibody Avastin to VEGF. As illustrated in FIG. 5, the inventors found that Avastin can bind to VEGF in complex with V_{L}D9. Thus, the inventors demonstrated that the V_{L} homodimers disclosed herein are able to bind to different epitopes than alternative antibody classes (such as IgG antibodies).

### Example 6

### Crystal Growth, Structure Determination and Refinement

The inventors next sought to further characterise the interaction between immunoglobulin homodimers of Ig5 and Ig12 respectively with the asymmetric antigen HEL as well as further characterise the structure of the immunoglobulin-antigen complexes of Ig5-HEL and Ig12-HEL respectively by producing crystals of the protein complexes and studying the complexes using X-ray crystallography.

Complexes of Ig52-HEL and Ig122-HEL were purified using size exclusion chromatography on a Superdex 75 10/300 GL size exclusion chromatography column (GE Healthcare). Peak fractions were collected and concentrated using 10 kDa Amicon Ultra microconcentrators (Millipore). Initial crystallization conditions were determined using a JCSG-plus crystal screen (Molecular Dimensions), a TTP Labtech Mosquito crystallization robot and 96-well MRC2 sitting drop crystallization plates (Swissci).

Crystals of the Ig52-HEL complex were grown at room temperature in a hanging-drop format by combining 2 μL of protein solution (at 6.5 mg/mL in PBS) with an equivalent volume of well solution (200 mM sodium formate, 18% (w/v) PEG 3,350). Crystals were cryoprotected by brief soaking in well solution doped with ethylene glycol to a concentration of 20% (v/v). Crystals were snap frozen in a nitrogen gas stream at 100 K and diffraction data was recorded using a Rigaku Micromax-007HF generator and MAR345dtb imaging plate (kindly provided by the University of Sydney). Crystals of the Ig12₂-HEL complex were grown at room temperature in a sitting-drop format by combining 0.4 μL of protein solution (at 5 mg/mL in PBS) with an equivalent volume of well solution (200 mM ammonium acetate, 100 mM BisTris (pH 5.5), 25% (w/v) PEG 3,350). Crystals were snap frozen without any further cryoprotection steps.

X-ray crystallography was performed in which the diffraction data was collected at 100 K on beamline MX2 at the Australian Synchrotron. Diffraction data obtained for Ig5₂-HEL complex and Ig12₂-HEL complex are shown in Table 2.

TABLE 2
Data collection and refinement statistics (molecular replacement) of the
crystal structures for Ig5-HEL and Ig12-HEL complexes respectively.
Diffraction Data

| | Dataset | |
|---|---|---|
| | Ig5₂-HEL | Ig12₂-HEL |
| Spacegroup | P2₁ | P6₃ |
| Unit cell dimensions: | 114.9, 39.4, 178.3; 106.4 | 126.8, 126.8, 40.7; 90 |
| a, b, c (Å); β (°) | | |
| Wavelength (Å) | 1.54179 | 0.95369 |
| Resolution range (Å) | 45.7-2.23 | 41.5-1.7 |
| Observed reflections^{$} | 447632 | 328598 |
| Unique reflections^{$} | 72711 | 41399 |
| Completeness^{$}* (%) | 93.5 (99.6) | 99.8 (99.5) |
| Multiplicity^{$}* | 6.2 (4.2) | 7.9 (6.5) |
| R [symbol] ^{$}* | 0.087 (0.29) | 0.098 (0.62) |
| Mean (I/sd)^{$}* | 14.3 (4.7) | 12.9 (2.8) |
| Wilson B (Å²) | 34.6 | 19.3 |

Refinement

| | | |
|---|---|---|
| Protein molecules/asu | 8 Ig, 4 HEL | 2 Ig, 1 HEL |
| Amino acids | 1391 | 330 |
| modeled/asu | | |
| Waters modeled/asu | 130 | 154 |
| Ramachandran^{#} | | |
| favored (%) | 96.2 | 96.3 |
| outliers (%) | 0.23 | 0.62 |
| R | 0.22 | 0.20 |
| R [symbol] (5% data) | 0.26 | 0.23 |
| RMSD bond lengths | 0.014 | 0.013 |
| (Å) | | |
| RMSD bond angles (°) | 1.45 | 1.38 |

Buried Surface^{@}

| | | |
|---|---|---|
| V_{L}dimer AB; | 592; 363, 467 | 706; 285, 406 |
| subunits-HEL (Å²) | | |
| V_{L}dimer CD; | 640; 409, 512 | — |
| subunits-HEL (Å²) | | |
| V_{L}dimer EF; | 617; 406, 458 | — |
| subunits-HEL (Å²) | | |
| V_{L}dimer GH; | 556; 372, 419 | — |
| subunits-HEL (Å²) | | |
| [D11.15 VH-VL; | — | — |
| subunits-PEL (Å²) | | |
| 750; 439, 200] | | |

Alternate side-chain conformers contacting HEL

| | | |
|---|---|---|
| V_{L} dimer AB | B-Tyr53 unresolved | — |
| | | B-Tyr94 alternative |
| V_{L} dimer CD | D-Tyr53 alternative | — |
| | | D-Tyr94 alternative |
| V_{L} dimer EF | — | — |
| V_{L} dimer GH | H-Tyr 94 unresolved | — |
| PDB entry | 4N1E | 4N1C |

^{$}As output by SCALA (Kunkel, 1985)
* Values in parentheses are of the highest resolution shell
^{#}As calculated by the MOLPROBITY validation server (Baatye et al. 2011)
^{@}As calculated by PDBePISA (Evans, 2006) [symbol]
indicates data missing or illegible when filed

Reflections were indexed and integrated with iMOSFLM according to the methods described in Battye et al. (2011), analysed for symmetry with POINTLESS according to the methods described in Evans (2006), scaled with SCALA according to the methods also described in Evans (2006), and imported into the CCP4i software package according to the methods as described in Winn et al. (2011) (Table 2). The structures were solved by molecular replacement using PHASER according to the methods as described in Emsley and Cowtan (2004). The search models for the Ig and HEL components were PDB entry 3UPA (κ light chain) and PDB entry 1ZVY (HEL), both stripped of side-chain moieties.

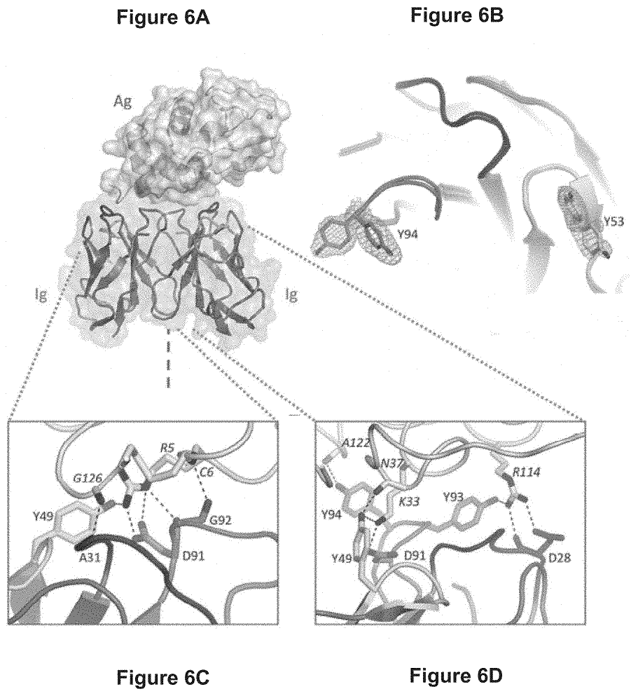

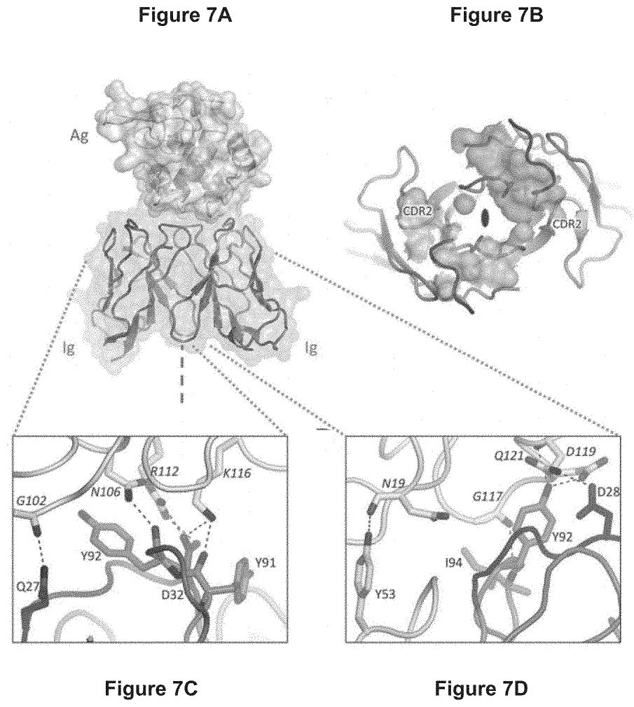

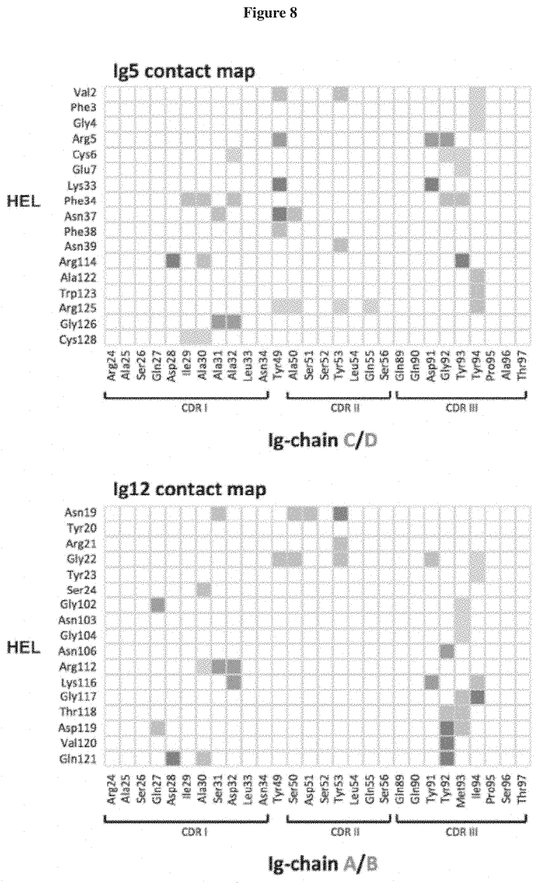

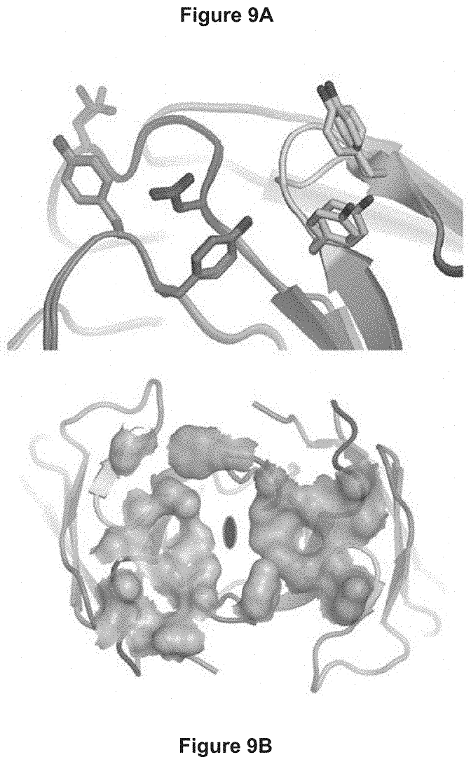

In both cases a 2:1 ratio of Ig:HEL was observed in the crystal; a pair of Ig monomers, arranged in Bence-Jones dimer configuration are bound to each HEL molecule for Ig5 (FIG. 6, FIG. 9) and Ig12 (FIG. 7, FIG. 9). Four essentially identical Ig2-HEL complexes were found in the asymmetric unit of the Ig5₂-HEL structure, where Ig dimers are chained AB, CD, EF, and GH, and corresponding HEL molecules are chained I, J, K and L (Table 3 and FIG. 8).

Non-crystallographic restraints were employed during early stages of refinement, although these were completely removed prior to the introduction of solvent molecules, and for the duration of latter stages of refinement. A single Ig₂-HEL complex was observed in the asymmetric unit of the Ig12₂-HEL structure.

TABLE 3
Ig5-HEL contact interface

| Residue | Chain | HEL contact | CC | RMSD (Å) |
|---|---|---|---|---|
| Asp28 | C | none | 0.93 | 0.42 |
| | D | H-bonds sidechain of Arg114 | 0.96 | |
| Tyr49 | C | H-bonds sidechain of Arg5 | 0.98 | 1.63 |
| | D | H-bonds mainchain of Asn37 | 0.98 | |
| Tyr53 | C | Packs against sidechain of Arg125 | 0.96 | 4.28 |
| | D | none | 0.93 | |
| Asp91 | C | H-bonds sidechain of Arg5 | 0.96 | 1.18 |
| | D | H-bonds sidechain of Lys33 | 0.96 | |
| Tyr93 | C | Packs against sidechain of Cys6 | 0.96 | 1.99 |
| | D | H-bonds sidechain of Arg114 | 0.96 | |
| Tyr94 | C | Packs against sidechain of Val2 | 0.97 | 4.87 |
| | D | Packs against sidechain of Trp123 | 0.97 | |

Real-space correlation coefficients (CC) for the 2Fo-Fc electron density map were calculated using Phenix according to the methods as described in Adams et al. (2010), proving a measure of how well the model fits the electron density on a residue basis. For Ig5 chains C and D the mean CC for all residues was 0.96 (ranging from 0.68-0.99). For interface CDR residues 28, 49, 91, 53, 93 and 94 the mean CC was also 0.96, strongly supporting the accuracy of the modeled side-chain conformations in these regions. The CC for these CDR residues in other Ig5 protomers was similarly high (0.95-0.96). Root mean square deviation (RMSD) between protomers for residues in the Ig5 structure were calculated using CCP4i according to the methods as described in Potterton et al. (2003). For interface CDR residues 28, 49, 91, 53, 93 and 94 the RMSD equalled 2.40 Å (side-chain atoms), while “core” residues (defined as those with a solvent accessible surface area less than 10%) displayed an RMSD of 0.28 Å using sidechain atoms of chains C and D. These results demonstrate large structural variation at CDR positions relative to a relatively rigid core.

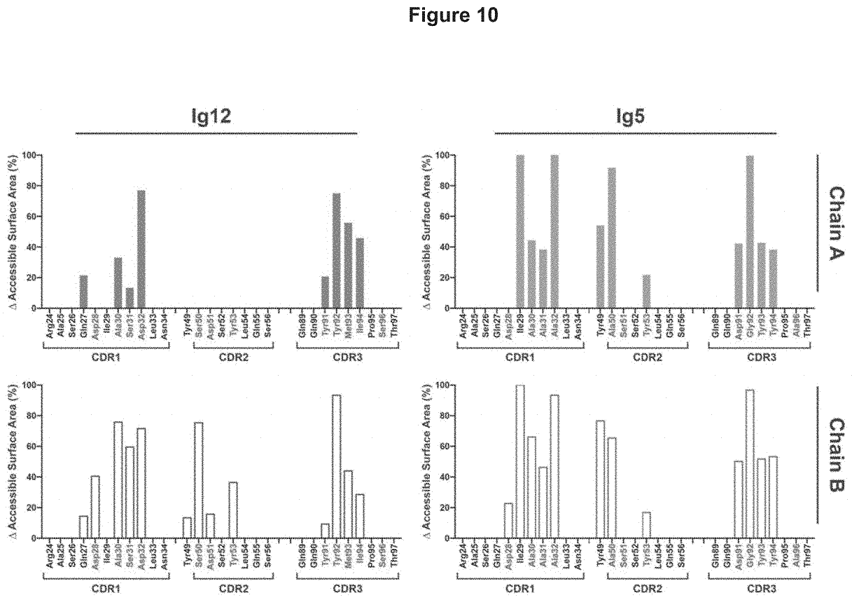

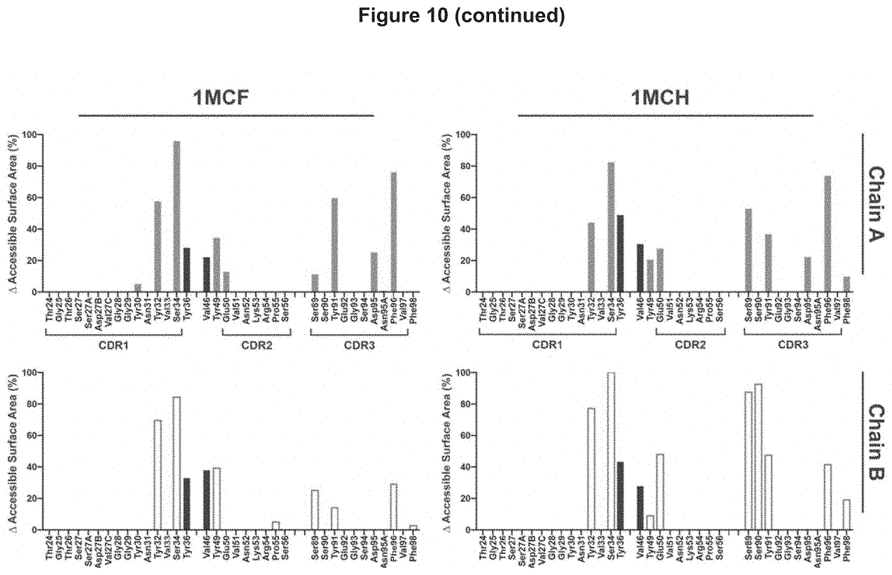

Initial rounds of rigid body refinement were followed by B-factor restrained refinement using REFMACS according to the methods of Murshudov et al. (1997), and employed torsion-libration-screw (TLS) parameterization according to the methods of Winn et al. (2011). During later stages of refinement water molecules were modeled into appropriate features of difference density. Models were compared with maps and manipulated in real space using COOT according to the methods of Emsley and Cowtan (2004). Models were scrutinized using the MOLPROBITY validation server according to the methods of Chen et al. (2010) (Table 1). Buried surfaces were calculated with PDBePISA according to the methods of Krissinel and Henrick (2007). Buried surface data are contained in Table 3, Table 4 and FIG. 10.

From the results presented in Table 3 and FIG. 6B, the inventors determined that in the Ig5₂-HEL structure, which contains four Ig5₂-HEL complexes in the asymmetric unit, superposition of the Ig domains within each complex reveals that the side chains of key residues adopt a range of different conformations (FIG. 6B). This conformational diversity is particularly pronounced for tyrosine residues at positions 53 and 94, which adopt strikingly different conformations (largely due to rotation about the chil dihedral angle, leaving the main chain fold unaffected) (FIG. 6B, Table 3). Hence, in the Ig5₂-HEL structure homodimer symmetry is relaxed through conformational side-chain diversity within CDR loops to complement the asymmetric antigen.

In contrast, such examples of structural plasticity are not observed in the Ig12₂-HEL structure, where superposition of the Ig protomers within the single complex in the asymmetric unit reveals few detectable differences in side chain conformations (FIG. 9A). Instead, a different mechanism based on the selective recruitment of CDR regions is dominant in this second structure. Here, each Ig subunit uses different combinations of contact residues, within otherwise structurally similar CDR loops, to form unique hydrogen-bonding networks with the HEL surfaces (FIG. 7C,D). The alternative utilization of otherwise identical CDR surfaces is particularly evident when mapping the contact-footprint of the antigen onto the two Ig protomers (FIG. 7B). This projection reveals selective usage of CDR2, with only one of the Ig12 subunits making extensive contacts through this CDR (detailed in FIG. 9). Such CDR contact selectivity is also observed in the Ig5₂-HEL complex (FIG. 6C,D and FIG. 9B), complementing conformational CDR side chain plasticity.

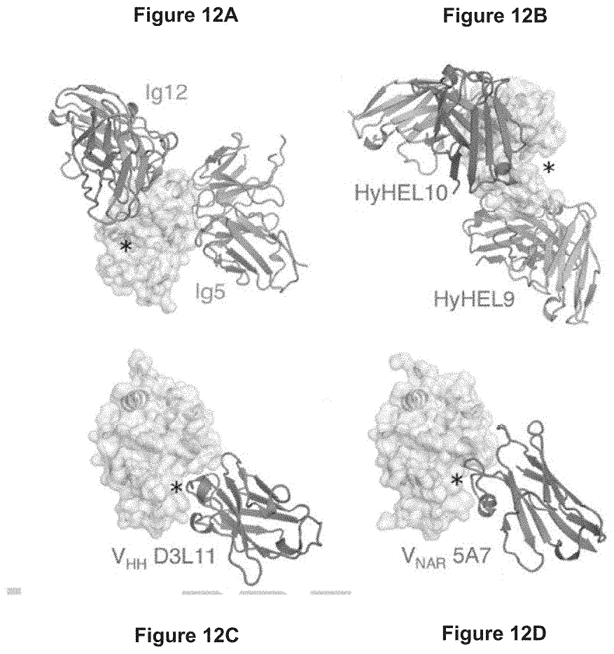

Surprisingly, the inventors also discovered that in both structures Ig5-HEL and Ig12-HEL, a receptor dimer is bound to a single HEL molecule, although the HEL epitopes targeted are completely different (FIG. 6B, FIG. 7B and Main FIG. 12A). Surprisingly, unlike previously reported Ig receptor structures in complex with antigen, in which V_{H} and V_{L} paralogues interact in a heterodimeric arrangement previously described in MacCallum et al. (1996), both Ig5-HEL and Ig12-HEL structures disclosed herein reveal a unique homodimeric nature of the receptor components.

Strikingly, the CDR regions of each receptor protomer, even though identical in amino acid sequence, form discrete and extensive contacts with different parts of the antigen surface (which lacks the two-fold rotational symmetry of the receptor). In the case of the Ig5₂-HEL structure, the Ig domains individually bury approximately 400 Å2 and 450 Å2 of the HEL surface (combined 850 Å2). Similar buried surface areas are observed for the Ig12₂-HEL structure (approximately 300 Å2, 400 Å2, and 700 Å2 combined). Indeed, the antigen contact surfaces are comparable in size to those of the Ig-Ig homodimer interfaces (at 600 Å2 and 700 Å2 for Ig5₂ and Ig12_{2,} respectively). Both receptors form large and well-defined interfaces (FIG. 8), similar in extent to what has been observed for heterodimeric antibody-antigen complexes (560-855 Å2 combined) previously described in Davies and Cohen (1996).

From Table 4, the inventors also surprisingly discover that the buried surface of 691-830 Å2 observed for the Ig12 and Ig5 complexes is much larger when compared to the buried surface areas for Bence Jones protein MCG towards a peptide which is only 462-557 Å2. This indicates that the immunoglobulin homodimers disclosed herein (exemplified by Ig12 and Ig5) bind much more strongly to a protein target than previously described Bence Jones proteins. Whilst these results are demonstrated with particular reference to Ig12 and Ig5, these two clones were selected for exemplification purposes only and other V_{L} homodimers made by the methods disclosed herein will likely demonstrate similar binding features to those described for Ig12 and Ig5.

The inventors have further characterized the type of interactions between Ig5 and Ig12 homodimers with the protein antigen. Surprisingly, the Ig5 and Ig12 structures outlined here are characterized by affinities that are orders of magnitude higher (nanomolar) and an extensive interface encompassing numerous hydrogen bond and charge interactions (Table 3, Table 4) when compared to the Bence Jones MCG protein, whose interaction with a protein are dominated by hydrophobic contacts, with limited hydrogen bonds and no salt bridges observed (Table 4). These results further confirm the strong interaction of immunoglobulin homodimers disclosed herein (exemplified by Ig5 and Ig12) to a protein antigen.

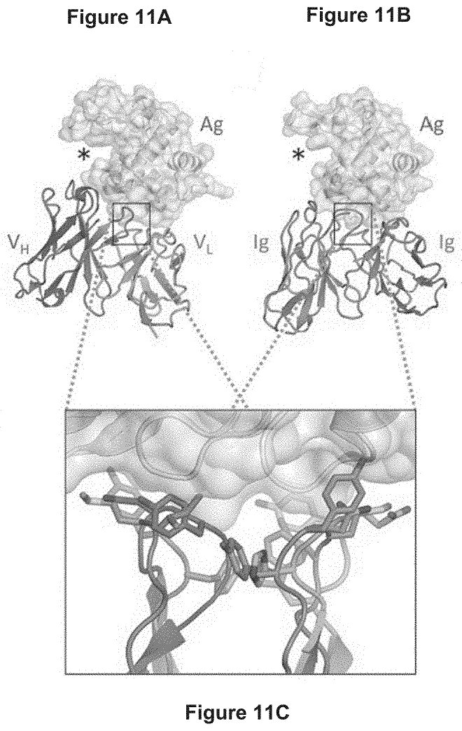

The inventors have also made the surprising observation that Ig12₂ homodimers bind distinctly outside the active site cleft of the HEL antigen (FIG. 11A,B—active site indicated by asterisks) while Ig5₂ targets a planar epitope distant from the catalytic site (FIG. 12A). This surprising observation is in stark contrast to single domain Ig formats, such as the heavy-chain only receptors of camelids, which only bind with strict 1:1 stoichiometry and a strong preference for targeting the active site cleft of HEL as shown in FIG. 12 (De Genst et al. 2006, Standfield et al. 2004). By binding away from the active cleft site and targeting a planar epitope away from the catalytic site, the Ig12₂ and Ig5₂ homodimers disclosed herein are able to overcome symmetry mismatch with an antigen via structural side-chain plasticity and differential recruitment of CDR loops. The Ig12₂ and Ig5₂ homodimers disclosed herein are able to bind specifically to asymmetric or symmetric antigens.

TABLE 4
Buried surface area and contact details for Ig5-HEL, Ig12-HEL, D11.15-PEL,
and MCG-peptide complexes.

| | Protein | | | | | | | | | |
|---|---|---|---|---|---|---|---|---|---|---|
| | VL12 | | VL5 | | D11.15 | | MCG | | MCG | |
| Name | Human VL | | Human VL | | Mouse Fab | | Human LC | | Human LC | |
| Framework | Vκ1 | | Vκ1 | | V_{H}1 | Vκ16 | Vλ2 | | Vλ2 | |
| PDB code | 4N1C | | 4N1E | | 1JHL | | 1MCB | | 1MCC | |
| Binding partner | HEL | | HEL | | PEL | | Ac-QfHp-OH | | Ac-QfHp-NH₂ | |
| Affinity (nM) | 64 | | 130 | | 15 | | | | | |
| Shape | 0.667 | | 0.725 | | 0.635 | | 0.633 | | 0.703 | |
| complementarity\hat{ } | | | | | | | | | | |
| Chain | A | B | A | B | H | L | A | B | A | B |
| Hydrogen bonds* | 6 | 6 | 8 | 6 | 7 | 2 | — | 1 | 1 | 1 |
| Salt bridges* | 1 | — | 1 | 2 | 2 | — | — | — | — | — |
| BSA antibody* | 691 | | 830 | | 638 | | 462 | | 385 | |
| Total per chain | 285 | 406 | 363 | 467 | 439 | 200 | 283 | 179 | 208 | 176 |
| CDR1 | 67 | 129 | 68 | 109 | 117 | 37 | 67 | 29 | 75 | 56 |
| CDR2 | | 82 | 56 | 31 | 93 | | 18 | 21 | 3 | 6 |
| CDR3 | 218 | 184 | 190 | 242 | 173 | 158 | 139 | 96 | 97 | 75 |
| Framework | | 11 | 49 | 85 | 56 | 5 | 59 | 33 | 33 | 39 |

| | Protein | | | | | | | |
|---|---|---|---|---|---|---|---|---|
| | MCG | | MCG | | MCG | | MCG | |
| Name | Human LC | | Human LC | | Human LC | | Human LC | |
| Framework | Vλ2 | | Vλ2 | | Vλ2 | | Vλ2 | |
| PDB code | 1MCD | | 1MCE | | 1MCF^{#} | | 1MCH^{#} | |
| Binding partner | Ac-fβHp-NH₂ | | Ac-QfHpβ-OH | | Ac-QfHpββ- | | Ac-QfHpββ- | |
| | | | OH | | | | OH | |
| Affinity (nM) | | | | | | | | |
| Shape | 0.537 | | 0.499 | | 0.671 | | 0.637 | |
| complementarity\hat{ } | | | | | | | | |
| Chain | A | B | A | B | A | B | A | B |
| Hydrogen bonds* | 1 | — | 3 | — | 3 | 2 | 4 | 2 |
| Salt bridges* | — | — | — | — | — | — | — | — |
| BSA antibody* | 353 | | 499 | | 530 | | 557 | |
| Total per chain | 157 | 196 | 297 | 202 | 306 | 224 | 284 | 273 |
| CDR1 | 36 | 72 | 93 | 81 | 88 | 94 | 64 | 63 |
| CDR2 | | 13 | 6 | 3 | 6 | 2 | 20 | 26 |
| CDR3 | 111 | 77 | 119 | 51 | 153 | 53 | 124 | 121 |
| Framework | 10 | 35 | 52 | 66 | 58 | 75 | 76 | 64 |

\hat{ }As calculated by Sc (Lefranc et al. 2009)
*Buried surface area as calculated by PDBePISA (Evans, 2006)
^{#}Peptides either soaked (1MCF) or co-crystallized (1MCH)
L amino acids capitalized
D amino acids in lower case
Ac: N-acetyl

NH2: amide

OH: carboxyl

Using the methods described above, the inventors sought to further characterise the interaction between immunoglobulin homodimer V_{L}D9 with the symmetrical antigen vascular endothelial growth factor A (VEGFA) as well as further characterise the structure of the immunoglobulin-antigen complex V_{L}D9-VEGFA by producing crystals of the protein complexes and studying the V_{L}D9-VEGFA complex using X-ray crystallography.

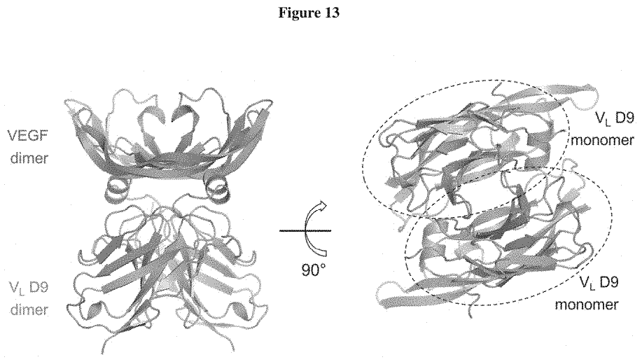

From the results obtained, the inventors discovered that each V_{L} of the homodimer V_{L}D9 contacts virtually identical surfaces of the VEGFA dimer (FIG. 13). The inventors determined that the intertwined cysteine knot arrangement of the VEGF dimer results in each V_{L} of V_{L}D9 contacting both VEGFA monomers, and the interaction is primarily mediated by hydrophobic and aromatic residues within the identical V_{L} domain CDR loops of V_{L}D9. Some solvent exposed VEGFA side chains exhibit conformational changes upon V_{L}D9 binding. The inventors observed no changes in side chain rotamers or differences in CDR loop conformations between each V_{L} of V_{L}D9.

### Example 7

### V_{L}D9 Homodimers and Anti-VEGF Antibody Avastin have Different VEGFA Epitopes

The inventors then sought to determine if the V_{L}D9 homodimer described herein binds to VEGFA using the same epitopes that would be bound by an anti-VEGF monoclonal antibody. The inventors used a commercially available anti-VEGF monoclonal antibody, Avastin. The inventors prepared Avastin-VEGFA complexes using generally the same methods described herein in Example 4. Crystals of Avastin-VEGFA were prepared using generally the same methods described herein in Example 6.

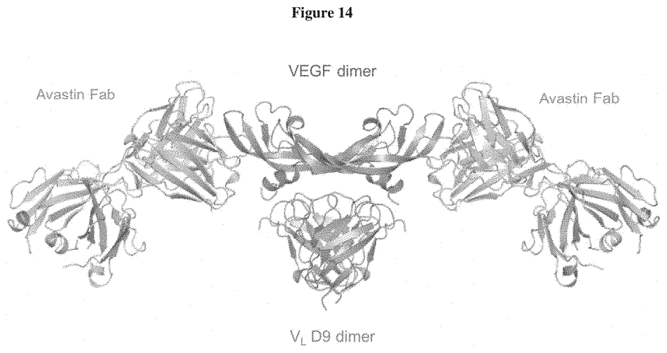

The inventors superimposed the crystal structure of V_{L}D9-VEGFA complex with Avastin-VEGFA complex. From the results obtained, the inventors determined that the epitopes of both V_{L}D9-VEGFA and Avastin-VEGFA complexes do not overlap (FIG. 14). This means that Avastin and V_{L}D9 homodimer described herein have different VEGF epitopes.

### Example 8

### Fusing V_{L}D9 to an Fc Does Not Alter VEGFA Interaction The inventors next sought to determine if the hinge and Fc domain interferes with the interaction of V_{L}D9 homodimer with VEGF.

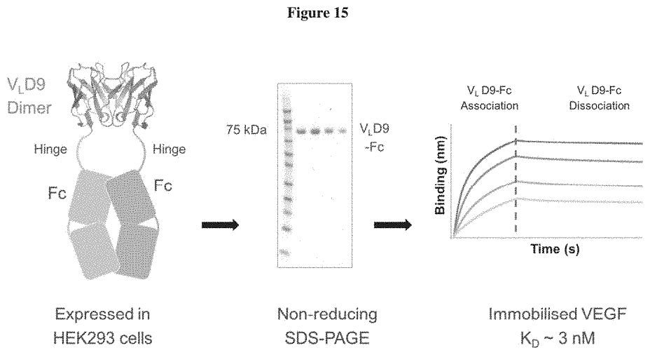

The inventors fused V_{L}D9 to the mouse IgG2c hinge and Fc region and expressed the fusion protein in Expi293 cells using known recombinant methods and in accordance with the manufacturer's instructions. The purified protein was then tested for binding to biotinylated VEGFA 121 using Biolayer interferometry (FIG. 15), which demonstrated that the hinge and Fc domain did not interfere with V_{L}D9 interaction with VEGF. VEGFA 121 was chosen as a representative example of all VEGF isoforms. VEGFA is part of the VEGF family, which includes VEGFB, VEGFC, VEGFD, VEGFE and PlGF. The family members bind to different sets of receptors and thus have different biological functions. However, each VEGF family member protein comprises a globular cysteine knot homodimer. Different VEGF isoforms vary in their heparin binding capacities, which are determined by the C-terminal region of the protein. For example, VEGFA 121 lacks heparin binding capacity (and is freely diffusible), while longer isoforms (e.g., VEGF 189 and VEGF 206) are almost completely bound to the extracellular matrix. VEGF165 has intermediary properties. Since VEGFA 121 essentially represents the minimal core of the protein, it was selected for use in the Biolayer interferometry assays disclosed herein.

### Example 9

### V_{L}D9-Fc Binds to Mouse VEGFA

The inventors next sought to test if the V_{L}D9-Fc fusion protein is able to bind to mouse VEGFA. To do this, the inventors tested mouse VEGFA164, human VEGFB and human PlGF for interaction with V_{L}D9-Fc using biolayer interferometry (VEGFC and VEGFD were not tested as they have glycosylation sites on the same side of the V_{L}D9 binding surface that would most likely prevent the interaction). While no binding was observed for VEGFB and PlGF, V_{L}D9-Fc bound to mouse VEGFA164 with an affinity of less than 10 nM, which was highly similar to that observed for human VEGFA 121. Thus, the immunoglobulin homodimers disclosed herein (e.g., produced by the methods disclosed herein) can retain their binding affinity for their specific antigen even when fused to an Fc domain.

### Example 10

### Homotetrameric V_{L}D9-Fc Binds to Two VEGFA Antigens

The inventors next fused the V_{L}D9 homodimer to a short hinge-Fc domain to determine if a V_{L}D9-Fc homotetramer would form and also to determine how many VEGFA antigens (which are in dimeric form) are able to be bound by the V_{L}D9-Fc homotetramers.

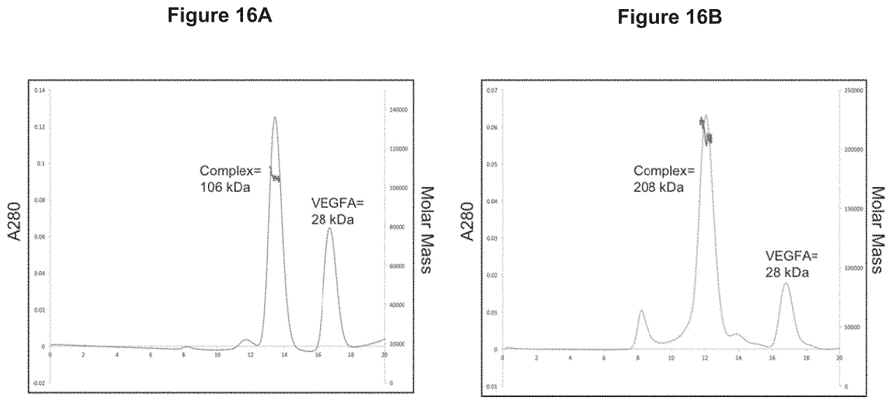

After fusing the V_{L}D9 homodimer to a short hinge-Fc domain using commonly known recombination techniques, the construct was then expressed in Expi 293 cells in accordance with manufacturer's instructions and the protein was purified as per the native hinge described above. Gel filtration analysis was performed using known methods in the art and the inventors determined that the fusion protein was more prone to aggregation than the construct that contained the native hinge. Mutating the hinge region using known techniques to produce a shorter hinge decreased the amount of aggregation observed (FIG. 16B), but the fusion protein was still poorly soluble at higher concentrations. Nonetheless, multi-angle laser light scattering (MALLS) analysis demonstrated that the heterotetramer bound to two VEGFA dimers (FIG. 16).

### Example 11

### V_{L}D9-Fc Constructs Do Not Affect the Proliferation of HUVEC Cells

To assess the effect of V_{L}D9-Fc fusions (both long native hinge and short mutant hinge constructs) on VEGFA binding to its receptors, the inventors performed proliferation assays in HUVEC cells. The inventors used VEGFA isoform 165 (VEGFA 165) because, besides VEGFA isoforms 121 and 189, VEGFA 165 is the most abundant and biologically active isoform. VEGFA 165 was therefore deemed to be the most suitable exemplary VEGF isoform to be used in the proliferation assays described herein.

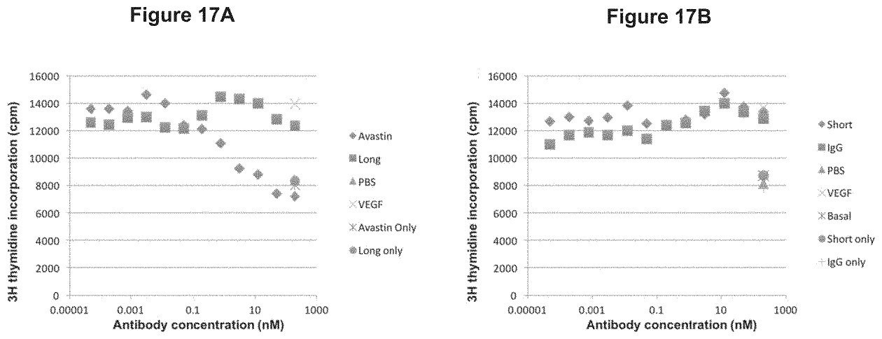

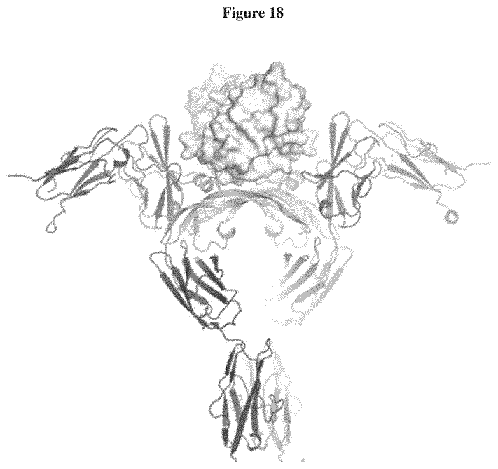

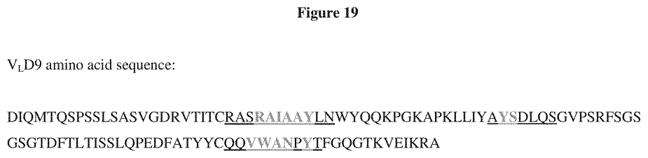

HUVEC cells were seeded (10 000 cells/ well) onto 96 well plates in basal media lacking growth factors. After ˜4 hours of recovery, VEGFA 165 was added with and without antibodies. The cells were then frown for 20 hrs, after which 0.5 μCi³H thymidine in basal media was added and the cells were harvested after an additional 20 hours. Unlike the decrease in proliferation observed for anti-VEGF antibody Avastin, which prevents VEGFA from binding to its receptors, the inventors determined that V_{L}D9-Fc constructs had no effect on the proliferation of HUVEC cells similar to the IgG controls (FIG. 17). This result suggests that V_{L}D9-Fc constructs do not interfere with VEGFA 165 binding to its receptors present on HUVEC cells, demonstrating that the immunoglobulin homodimers disclosed herein can bind with high affinity and specificity to their antigens without interfering with the normal function of those antigens.

The inventors observed no steric clashes between VEGFR1 and the V_{L}D9 homodimer when the structures of the two complexes were superimposed onto one another (FIG. 18). This result suggests that V_{L}D9 homodimer can most likely interact with VEGFA when bound to its receptors. This result is also consistent with proliferation assay results which demonstrated that V_{L}D9-Fc fusions did not affect the proliferation of HUVEC cells, in contrast with anti-VEGF antibody Avastin (which prevents VEGFA from binding to its receptors).

### REFERENCES

Adams P D, et al. (2010) PHENIX: a comprehensive Python-based system for macromolecular structure solution Acta Crystallogr D Biol Crystallogr 66:213-221.
Ausubel F. M. et al. (editors), Current Protocols in Molecular Biology, Greene Pub. Associates and Wiley-Interscience (1988, including all updates until present);
Al-Lazikani et al. (1997) Standard conformations for the canonical structures of immunoglobulins J Mol Biol 273: 927-948.
Battye T G, Kontogiannis L, Johnson O, Powell H R, & Leslie A G (2011) iMOSFLM: a new graphical interface for diffraction-image processing with MOSFLM Acta Crystallogr D Biol Crystallogr 67:271-281.
Bernier G M and Putnam F W (1963) Monomer - Dimer forms of Bence Jones Proteins Nature 200:223-225.
Borrebaeck (ed), Antibody Engineering, Oxford University Press, 1995 (ISBNO195091507).
Carter et al. (1985) Improved oligonucleotide site-directed mutagenesis using M13 vectors Nucleic Acids Res. 13:4431-4443, 1985
Chen V B, et al. (2010) MolProbity: all-atom structure validation for macromolecular crystallography Acta Crystallogr D Biol Crystallogr 66:12-21.
Chothia C A M and Lesk J (1987) Canonical structures for the hypervariable regions of immunoglobulins Mol Biol 196:901 -917.
Chothia et al. (1989) Conformations of immunoglobulin hypervariable regions Nature 342:877-883.
Davies D R & Cohen G H (1996) Interactions of protein antigens with antibodies Proc Natl Acad Sci USA 93:7-12.
De Genst E, et al. (2006) Molecular basis for the preferential cleft recognition by dromedary heavy-chain antibodies Proc Natl Acad Sci USA 103:4586-4591.
Edmundson A B, et al. (1993) Principles and pitfalls in designing site-directed peptide ligands Proteins 16:246-267.
Edmundson A B and Manion C V (1998) Treatment of osteoarthritis with aspartame Clin Pharmacol Ther 63:580-593
Emsley P and Cowtan K (2004) Coot: model-building tools for molecular graphics Acta Crystallogr D Biol Crystallogr 60:2126-2132.
Evans P (2006) Scaling and assessment of data quality Acta Crystallogr D Biol Crystallogr 62:72-82.
Goding, (1986) Monoclonal Antibodies: Principles and Practice, Academic Press, pp. 59-103;
Hamers-Casterman C, Atarhouch T, Muyldermans S, Robinson G, Hammers C, Songa E B, et al. (1993) Naturally occurring antibodies devoid of light chains Nature 363: 446-448
Ho et al. (1989) Site-directed mutagenesis by overlap extension using the polymerase chain reaction Gene 77:51-59, 1989
Hoogenboom H R, et al. (1991) Multi-subunit proteins on the surface of filamentous phage: methodologies for displaying antibody (Fab) heavy and light chains Nucleic Acids Res 19:4133-4137;
Jones S and Thomton J M. (1995a) Protein-protein interactions: A review of protein dimer structures. Prog Biophys Mol Biol 63:31-65.
Jones S and Thornton J M. (1995b) Principles of protein-protein interactions. Proc Natl Acad Sei USA 93:13-20.
Krissinel E and Henrick K (2007) Inference of macromolecular assemblies from crystalline state J Mol Biol 372:774-797.
Kohler G and Milstein C (1975) Continuous cultures of fused cells secreting antibody of predefined specificity Nature 256:495-497.
Kunkel T A (1985) Rapid and efficient site-specific mutagenesis without phenotypic selection Proceedings of the National Academy of Sciences 82:488-492.
Largaespada et al. (1990) The activity of an ABL-MYC retrovirus in fibroblast cell lines and in lymphocytes Curr. Top. Microbiol. Immunol 166: 91-96.
Lee C M, lorno N, Sierro F, & Christ D (2007) Selection of human antibody fragments by phage display Nat Protoc 2:3001-3008.
Lefranc M P, et al. (2009) IMGT, the international ImMunoGeneTics information system Nucleic Acids Res 37:D1006-1012.
Lewis R A. (1991) Clefts and binding sites in protein receptors Methods Enzymol 202:126-156.
MacCallum R M, Martin A C, & Thornton J M (1996) Antibody-antigen interactions: contact analysis and binding site topography J Mol Biol 262:732-745.
Murshudov G N, Vagin A A, & Dodson E J (1997) Refinement of macromolecular structures by the maximum-likelihood method Acta Crystallogr D Biol Crystallogr 53:240-255.
Pluckthun in The Pharmacology of Monoclonal Antibodies, vol. 113, Rosenburg and Moore eds., Springer Verlag, New York, pp. 269-315, 1994
Potterton E, Briggs P, Turkenburg M, & Dodson E (2003) A graphical user interface to the CCP4 program suite Acta Crystallogr D Biol Crystallogr 59:1131-1137.
Rouet R, Dudgeon K, & Christ D (2012) Generation of human single domain antibody repertoires by Kunkel mutagenesis Methods Mol Biol 907:195-209.
Sambrook et al. (1989) Molecular Cloning: A Laboratory Manual, Cold Spring Harbour Laboratory Press.
Skerra, A (1993) Bacterial expression of immunoglobulin fragments Curr Opin Immunol 5(2): 256-62
Stanfield R L, Dooley H, Flajnik M F, and Wilson I A (2004) Crystal structure of a shark single-domain antibody V region in complex with lysozyme Science 305:1770-1773.
Weissinger et al. (1991) Induction of plasmacytomas secreting antigen-specific monoclonal antibodies with a retrovirus expressing v-abl and c-myc Proc. Natl. Acad. Sci USA, 88:8735-8739.
Winn M D, et al. (2011) Overview of the CCP4 suite and current developments Acta Crystallogr D Biol Crystallogr 67:235-242.
Ye H (2006) Simultaneous determination of protein aggregation, degradation, and absolute molecular weight by size exclusion chromatography—multiangle laser light scattering Analytical Biochemistry 356:76-85.

1. A method for screening a plurality of immunoglobulin homodimers, each homodimer comprising two identical light chain variable domain monomers, wherein screening of the immunoglobulin homodimers comprises identifying homodimers that are capable of binding specifically to an antigen to form an immunoglobulin-antigen complex.

2. The method of claim 1, wherein the plurality of immunoglobulin homodimers are prepared by providing a plurality of light chain variable domain monomers under conditions that allow any of the light chain variable domain monomers that are capable of dimerizing, to dimerize.

3. The method of claim 2, comprising expressing each light chain variable domain monomer in a bacteriophage that is capable of presenting multiple copies of the light chain variable domain monomer on its surface.

4. The method of any preceding claim, which comprises performing:
(i) an affinity screening step; and
(ii) a dimerization screening step.

5. The method of claim 4, wherein the affinity screening step comprises contacting the plurality of immunoglobulin homodimers with the antigen and selecting those immunoglobulin homodimers that bind specifically to the antigen.

6. The method of claim 4 or claim 5, wherein the dimerization screening step comprises determining the stoichiometric ratio of immunoglobulin monomer to antigen in the immunoglobulin-antigen complex.

7. The method of claim 6, which comprises selecting an immunoglobulin homodimer when the stoichiometric ratio of immunoglobulin monomer to antigen in the immunoglobulin-antigen complex is about 2:1.

8. The method of claim 6 or claim 7, wherein the stoichiometric ratio of immunoglobulin monomer to antigen in the immunoglobulin-antigen complex is determined by:
(i) measuring the quantity of any two or more of the immunoglobulin homodimer, antigen and immunoglobulin-antigen complex in a mixture containing the immunoglobulin homodimer and the antigen; and
(ii) comparing the relative quantities of any two or more of the immunoglobulin homodimer, antigen and immunoglobulin-antigen complex measured in (i) to determine the stoichiometric ratio of immunoglobulin monomer to antigen in the immunoglobulin-antigen complex.

9. The method of claim 8, wherein (i) further comprises measuring the quantity of unbound immunoglobulin monomer, unbound antigen, and/or unbound immunoglobulin homodimer in the mixture.

10. The method of any one of claims 6 to 9, comprising separating the immunoglobulin homodimer, antigen and immunoglobulin-antigen complex.

11. The method of claim 10, wherein the immunoglobulin homodimer, antigen and immunoglobulin-antigen complex are separated by chromatography.

12. The method of claim 11, wherein the chromatography is size exclusion chromatography.

13. The method of any one of claims 10 to 12, wherein the stoichiometric ratio of immunoglobulin to antigen is determined using an apparatus comprising particles or a matrix for separating compounds of different molecular weight, whereby said compounds are eluted from said apparatus in order of descending molecular weight.

14. The method of claim 13, comprising:
contacting the apparatus with a mixture containing a predetermined amount of the light chain variable domain monomer or the immunoglobulin homodimer, and containing a predetermined amount of the antigen; and
eluting from the apparatus the light chain variable domain monomer and/or the immunoglobulin homodimer;
eluting from the apparatus any unbound antigen; and/or eluting from the apparatus the immunoglobulin-antigen complex.

15. The method of claim 14, comprising incubating the mixture for at least 1 hour at room temperature prior to contact with the apparatus.

16. The method of any one of claims 8-15, wherein the measuring comprises measuring by light scattering.

17. The method of claim 16, which comprises the use of a light scattering photometer with an interferometric refractometer.

18. The method of any one of claims 4 to 17, wherein the affinity screening step is performed using an optical analytical technique for measuring biomolecular interactions.

19. The method of claim 18, which comprises any one of dual polarisation interferometry, static light scattering, dynamic light scattering, surface plasmon resonance, fluorescence polarisation/anisotropy, fluorescence correlation spectroscopy or nuclear magnetic resonance.

20. The method of any one of claims 4 to 19, wherein the affinity screening step comprises measuring the binding kinetics, binding specificity, rates of association and dissociation, or concentration of bound components, of the immunoglobulin-antigen complex.

21. The method of any one of claims 4-20, wherein the affinity screening step comprises measuring the association constant (Kₐ), dissociation constant (K_{d}) and/or equilibrium binding constant (K_{D}).

22. The method of any preceding claim, comprising selecting a homodimer having nanomolar affinity for the antigen.

23. The method of any preceding claim, comprising selecting a homodimer which binds to a planar surface on the antigen or a cleft within the surface of the antigen.

24. The method of any preceding claim, comprising selecting a homodimer which covers a surface area of the antigen of over 600 Å².

25. The method of any preceding claim, wherein the light chain variable domain monomers of the homodimer comprise one or more tyrosine residues in one or more complementarity determining regions.

26. The method of any preceding claim, wherein the light chain variable domain monomers are provided by expressing a polynucleotide comprising human light chain variable region V and J segments.

27. The method of any preceding claim, wherein variation in the plurality of immunoglobulin homodimers is provided by performing mutagenesis.

28. An immunoglobulin homodimer identified and/or selected by the method of any preceding claim.


SEQUENCE LISTING (23 sequences)

SEQ ID NO: 1 - protein, 108 aa, Artificial Sequence
  Amino acid sequence diversity for immunoglobulin Ig5
  MISC_FEATURE (28)..(28): Where Xaa is Tyr, Ser, Ala or Asp
  MISC_FEATURE (30)..(32): Where Xaa is Tyr, Ser, Ala or Asp
  MISC_FEATURE (50)..(51): Where Xaa is Tyr, Ser, Ala or Asp
  MISC_FEATURE (53)..(53): Where Xaa is Tyr, Ser, Ala or Asp
  MISC_FEATURE (91)..(94): Where Xaa is Tyr, Ser, Ala , Asp, Gly, Ile, Leu, Pro, Arg, Thr, Val, Glu, Phe, His, Lys, Met, Asn, Gln or Trp
  MISC_FEATURE (96)..(96): Where Xaa is Tyr, Ser, Ala, Asp, Gly, Ile, Leu, Pro, Arg, Thr, Val, Glu, Phe, His, Lys, Met, Asn, Gln or Trp
DIQMTQSPSSLSASVGDRVTITCRASQXIXXXLNWYQQKPGKAPKLLIYXXSXLQSGVPSRFSGSGSGTDFTLTISSLQPEDFATYYCQQXXXXPXTFGQGTKVEIKR

SEQ ID NO: 2 - protein, 11 aa, Artificial Sequence
  Amino acid sequence diversity for CDR1 of immunoglobulin Ig5
  MISC_FEATURE (5)..(5): Where Xaa is Tyr, Ser, Ala or Asp
  MISC_FEATURE (7)..(9): Where Xaa is Tyr, Ser, Ala or Asp
RASQXIXXXLN

SEQ ID NO: 3 - protein, 7 aa, Artificial Sequence
  Amino acid sequence diversity for CDR2 of immunoglobulin Ig5
  MISC_FEATURE (1)..(2): Where Xaa is Try, Ser, Ala or Asp
  MISC_FEATURE (4)..(4): Where Xaa is Try, Ser, Ala or Asp
XXSXLQS

SEQ ID NO: 4 - protein, 9 aa, Artificial Sequence
  Amino acid sequence diversity for CDR3 of immunoglobulin Ig5
  MISC_FEATURE (3)..(6): Where Xaa is Tyr, Ser, Ala, Asp, Gly, Ile, Leu, Pro, Arg, Thr, Val, Glue, Phe, His, Lys, Met, Asn, Gln or Trp
  MISC_FEATURE (8)..(8): Where Xaa is Tyr, Ser, Ala, Asp, Gly, Ile, Leu, Pro, Arg, Thr, Val, Glue, Phe, His, Lys, Met, Asn, Gln or Trp
QQXXXXPXT

SEQ ID NO: 5 - protein, 108 aa, Artificial Sequence
  Amino acid sequence for immunoglobulin Ig5 cluster family
DIQMTQSPSSLSASVGDRVTITCRASQDIAAALNWYQQKPGKAPKLLIYASSYLQSGVPSRFSGSGSGTDFTLTISSLQPEDFATYYCQQDGYYPATFGQGTKVEIKR

SEQ ID NO: 6 - protein, 10 aa, Artificial sequence
  Amino acid sequence for CDR1 of immunoglobulin Ig5 cluster family
RASQDIAAAL

SEQ ID NO: 7 - protein, 7 aa, Artificial sequence
  Amino acid sequence for CDR2 of immunoglobulin Ig5 cluster family
ASSYLQS

SEQ ID NO: 8 - protein, 9 aa, Artificial sequence
  Amino acid sequence for CDR3 of immunoglobulin Ig5 cluster family
QQDGYYPAT

SEQ ID NO: 9 - protein, 108 aa, Artificial Sequence; identical to SEQ ID NO: 1
  Amino acid sequence diversity for immunoglobulin Ig12
  MISC_FEATURE (28)..(28): Where Xaa is Tyr, Ser, Ala or Asp
  MISC_FEATURE (30)..(32): Where Xaa is Tyr, Ser, Ala or Asp
  MISC_FEATURE (50)..(51): Where Xaa is Tyr, Ser, Ala or Asp
  MISC_FEATURE (53)..(53): Where Xaa is Tyr, Ser, Ala or Asp
  MISC_FEATURE (91)..(94): Where Xaa is Tyr, Ser, Ala, Asp, Gly, Ile, Leu, Pro, Arg, Thr, Val, Glu, Phe, His, Lys, Met, Asn, Gln or Trp
  MISC_FEATURE (91)..(94): Where Xaa is Tyr, Ser, Ala, Asp, Gly, Ile, Leu, Pro, Arg, Thr, Val, Glu, Phe, His, Lys, Met, Asn, Gln or Trp
  MISC_FEATURE (96)..(96): Where Xaa is Tyr, Ser, Ala, Asp, Gly, Ile, Leu, Pro, Arg, Thr, Val, Glu, Phe, His, Lys, Met, Asn, Gln or Trp

SEQ ID NO: 10 - protein, 11 aa, Artificial Sequence
  Amino acid sequence diversity for CDR1 of immunoglobulin Ig12
  MISC_FEATURE (5)..(5): Where Xaa is Try, Ser, Ala or Asp
  MISC_FEATURE (7)..(9): Where Xaa is Try, Ser, Ala or Asp
RASQXIXXXLN

SEQ ID NO: 11 - protein, 7 aa, Artificial Sequence
  Amino acid sequence diversity for CDR2 of immunoglobulin Ig12
  MISC_FEATURE (1)..(2): Where Xaa is Try, Ser, Ala or Asp
  MISC_FEATURE (4)..(4): Where Xaa is Try, Ser, Ala or Asp
XXSXLQS

SEQ ID NO: 12 - protein, 9 aa, Artificial Sequence
  Amino acid sequence diversity for CDR3 of immunoglobulin Ig12
  MISC_FEATURE (3)..(6): Where Xaa is Tyr, Ser, Ala, Asp, Gly, Ile, Leu, Pro, Arg, Thr, Val, Glue, Phe, His, Lys, Met, Asn, Gln or Trp
  MISC_FEATURE (8)..(8): Where Xaa is Tyr, Ser, Ala, Asp, Gly, Ile, Leu, Pro, Arg, Thr, Val, Glue, Phe, His, Lys, Met, Asn, Gln or Trp
QQXXXXPXT

SEQ ID NO: 13 - protein, 108 aa, Artificial Sequence
  Amino acid sequence of immunoglobulin Ig12 cluster family
DIQMTQSPSSLSASVGDRVTITCRASQDIASDLNWYQQKPGKAPKLLIYSDSYLQSGVPSRFSGSGSGTDFTLTISSLQPEDFATYYCQQYYMIPSTFGQGTKVEIKR

SEQ ID NO: 14 - protein, 11 aa, Artificial sequence
  Amino acid sequence for CDR1 of immunoglobulin Ig12 cluster family
RASQDIASDLN

SEQ ID NO: 15 - protein, 7 aa, Artificial sequence
  Amino acid sequence for CDR2 of immunoglobulin Ig12 cluster family
SDSYLQS

SEQ ID NO: 16 - protein, 9 aa, Artificial sequence
  Amino acid sequence for CDR3 of immunoglobulin Ig12 cluster family
QQYYMIPST

SEQ ID NO: 17 - DNA, 59 bp, Artificial Sequence
  Nucleic acid sequence diversity of oligonucleotide for CDR1
  misc_feature (23)..(23): Where n is T or G
  misc_feature (24)..(24): Where n is A or C
  misc_feature (29)..(29): Where n is T or G
  misc_feature (30)..(30): Where n is A or C
  misc_feature (32)..(32): Where n is T or G
  misc_feature (33)..(33): Where n is A or C
  misc_feature (35)..(35): Where n is T or G
  misc_feature (36)..(36): Where n is A or C
catcacttgccgggcaagtcagnntattnntnntnntttaaattggtatcagcagaaac

SEQ ID NO: 18 - DNA, 64 bp, Artificial Sequence
  Nucleic acid sequence diversity of oligonucleotide for CDR2
  misc_feature (30)..(30): Where n is T or G
  misc_feature (31)..(31): Where n is A or C
  misc_feature (33)..(33): Where n is T or G
  misc_feature (34)..(34): Where n is A or C
  misc_feature (39)..(39): Where n is T or G
  misc_feature (40)..(40): Where n is A or C
cagggaaagcccctaagctcctgatctatnntnnttccnntttgcaaagtggggtcccatcaag

SEQ ID NO: 19 - DNA, 57 bp, Artificial Sequence
  Nucleic acid sequence diversity of oligonucleotide for CDR3
  misc_feature (19)..(30): Where n is T, G, A or C
  misc_feature (34)..(36): Where n is T, G, A or C
acttactactgtcaacagnnnnnnnnnnnncctnnnacgttcggccaagggaccaag

SEQ ID NO: 20 - protein, 109 aa, Artificial sequence
  Amino acid sequence for immunoglobulin VLD9
DIQMTQSPSSLSASVGDRVTITCRASRAIAAYLNWYQQKPGKAPKLLIYAYSDLQSGVPSRFSGSGSGTDFTLTISSLQPEDFATYYCQQVWANPYTFGQGTKVEIKRA

SEQ ID NO: 21 - protein, 12 aa, Artificial sequence
  Amino acid sequence for CDR1 of immunoglobulin VLD9
CRASRAIAAYLN

SEQ ID NO: 22 - protein, 7 aa, Artificial sequence
  Amino acid sequence for CDR2 of immunoglobulin VLD9
AYSDLQS

SEQ ID NO: 23 - protein, 9 aa, Artificial sequence
  Amino acid sequence for CDR3 of immunoglobulin VLD9
QQVWANPYT